SEQUENCE LISTING (56 sequences)

SEQ ID NO: 1 - protein, 13 aa, Artificial sequence
  Synthetic
NWYLPWLGTNDWC

SEQ ID NO: 2 - protein, 17 aa, Artificial sequence
  Synthetic
NWYLPWLGTNDWGGGSC

SEQ ID NO: 3 - protein, 16 aa, Artificial sequence
  Synthetic
NWYLPWLGTNDWGGGK

SEQ ID NO: 4 - protein, 12 aa, Artificial sequence
  Synthetic
NWYGAGAGTNDW

SEQ ID NO: 5 - protein, 13 aa, Artificial sequence
  Synthetic
GALDTTHHRPWTC

SEQ ID NO: 6 - protein, 12 aa, Artificial sequence
  Synthetic
GAGAAGGAGGGG

SEQ ID NO: 7 - protein, 16 aa, Artificial sequence
  Synthetic
GALDTTHHRPWTGGGK

SEQ ID NO: 8 - protein, 33 aa, Artificial sequence
  Synthetic
NWYLPWLGTNDWAGILARNLVPMVATVQGQNLC

SEQ ID NO: 9 - protein, 33 aa, Artificial sequence
  Synthetic
GAGAAGGAGGGGAGILARNLVPMVATVQGQNLC

SEQ ID NO: 10 - protein, 60 aa, Artificial sequence
  Synthetic
NWYLPWLGTNDWGCFTWPPWQAGILARNLVPMVATVQGQNLKYQEFFWDANDIYRIFAEL

SEQ ID NO: 11 - protein, 60 aa, Artificial sequence
  Synthetic
GALDTTHHRPWTGCFTWPPWQAGILARNLVPMVATVQGQNLKYQEFFWDANDIYRIFAEL

SEQ ID NO: 12 - RNA, 19 nt, Artificial sequence
  Synthetic
gcuccaugaugcguuaucu

SEQ ID NO: 13 - protein, 6 aa, Artificial sequence
  Synthetic
XLPWLX

SEQ ID NO: 14 - protein, 8 aa, Artificial sequence
  Synthetic
XWYLPWLG

SEQ ID NO: 15 - protein, 12 aa, Artificial sequence
  Synthetic
XWYLPWLGTNDW

SEQ ID NO: 16 - protein, 8 aa, Artificial sequence
  Synthetic
NWYLPWLG

SEQ ID NO: 17 - protein, 12 aa, Artificial sequence
  Synthetic
NWYLPWLGTNDW

SEQ ID NO: 18 - protein, 7 aa, Artificial sequence
  Synthetic
LTVSPWY

SEQ ID NO: 19 - protein, 9 aa, Artificial sequence
  Synthetic
NLVPMVATV

SEQ ID NO: 20 - protein, 4 aa, Artificial sequence
  Synthetic
GGGS

SEQ ID NO: 21 - protein, 7 aa, Artificial sequence
  Synthetic
GGGSRRR

SEQ ID NO: 22 - protein, 9 aa, Artificial sequence
  Synthetic
ILKEPVHGV

SEQ ID NO: 23 - protein, 15 aa, Artificial sequence
  Synthetic
AGILARNLVPMVATV

SEQ ID NO: 24 - protein, 11 aa, Artificial sequence
  Synthetic
FFWDANDIYRI

SEQ ID NO: 25 - protein, 12 aa, Artificial sequence
  Synthetic
QWELPWLMQPPL

SEQ ID NO: 26 - protein, 12 aa, Artificial sequence
  Synthetic
SPGLSLVSHMQT

SEQ ID NO: 27 - protein, 7 aa, Artificial sequence
  Synthetic
QLPRTAL

SEQ ID NO: 28 - protein, 7 aa, Artificial sequence
  Synthetic
GETRAPL

SEQ ID NO: 29 - protein, 9 aa, Artificial sequence
  Synthetic
NWXLXWLXW

SEQ ID NO: 30 - protein, 12 aa, Artificial sequence
  Synthetic
NWXLPWLGTNDW

SEQ ID NO: 31 - protein, 12 aa, Artificial sequence
  Synthetic
NWYLXWLGTNDW

SEQ ID NO: 32 - protein, 12 aa, Artificial sequence
  Synthetic
NWYLPWLXTNDW

SEQ ID NO: 33 - protein, 12 aa, Artificial sequence
  Synthetic
NWYLPWLGXNDW

SEQ ID NO: 34 - protein, 12 aa, Artificial sequence
  Synthetic
NWYLPWLGTXDW

SEQ ID NO: 35 - protein, 12 aa, Artificial sequence
  Synthetic
NWYLPWLGTNXW

SEQ ID NO: 36 - protein, 11 aa, Artificial sequence
  Synthetic
NWYLPWLGTNW

SEQ ID NO: 37 - protein, 11 aa, Artificial sequence
  Synthetic
NWYLPWLGTDW

SEQ ID NO: 38 - protein, 10 aa, Artificial sequence
  Synthetic
NWYLPWLGTW

SEQ ID NO: 39 - protein, 11 aa, Artificial sequence
  Synthetic
NWYPWLGTNDW

SEQ ID NO: 40 - protein, 11 aa, Artificial sequence
  Synthetic
NWYLPWGTNDW

SEQ ID NO: 41 - protein, 10 aa, Artificial sequence
  Synthetic
NWYPWGTNDW

SEQ ID NO: 42 - protein, 9 aa, Artificial sequence
  Synthetic
RRRRRRRRR

SEQ ID NO: 43 - protein, 5 aa, Artificial sequence
  Synthetic
GGGSC

SEQ ID NO: 44 - protein, 12 aa, Artificial sequence
  Synthetic
AWYLPWLGTNDW

SEQ ID NO: 45 - protein, 12 aa, Artificial sequence
  Synthetic
NAYLPWLGTNDW

SEQ ID NO: 46 - protein, 12 aa, Artificial sequence
  Synthetic
NWALPWLGTNDW

SEQ ID NO: 47 - protein, 12 aa, Artificial sequence
  Synthetic
NWYAPWLGTNDW

SEQ ID NO: 48 - protein, 12 aa, Artificial sequence
  Synthetic
NWYLAWLGTNDW

SEQ ID NO: 49 - protein, 12 aa, Artificial sequence
  Synthetic
NWYLPALGTNDW

SEQ ID NO: 50 - protein, 12 aa, Artificial sequence
  Synthetic
NWYLPWAGTNDW

SEQ ID NO: 51 - protein, 12 aa, Artificial sequence
  Synthetic
NWYLPWLATNDW

SEQ ID NO: 52 - protein, 12 aa, Artificial sequence
  Synthetic
NWYLPWLGANDW

SEQ ID NO: 53 - protein, 12 aa, Artificial sequence
  Synthetic
NWYLPWLGTADW

SEQ ID NO: 54 - protein, 12 aa, Artificial sequence
  Synthetic
NWYLPWLGTNAW

SEQ ID NO: 55 - protein, 12 aa, Artificial sequence
  Synthetic
NWYLPWLGTNDA

SEQ ID NO: 56 - protein, 12 aa, Artificial sequence
  Synthetic
NWYLPWLGTAAW

(19) United States
(12) Patent Application Publication
(10) Pub. No.: US 2016/0083424 A1
(43) Pub. Date: Mar. 24, 2016

(54) COMPOSITIONS AND METHODS FOR TARGETING ANTIGEN-PRESENTING CELLS

(71) Applicant: OSLO UNIVERSITETSSYKEHUS HF, Oslo (NO)

(72) Inventors: Mouldy Sioud, Oslo (NO); Gjertrud Skorstad, Oslo (NO)

(21) Appl. No.: 14/786,435

(22) PCT Filed: Apr. 24, 2014
(86) PCT No.: PCT/IB2014/001731

§ 371 Date: Oct. 22, 2015

Related U.S. Application Data
(60) Provisional application No. 61/816,299, filed on Apr. 26, 2013.

(51) Int. Cl. C07K 7/06; G01N 33/569; C07K 7/08

(52) U.S. Cl. CPC C07K 7/06; C07K 7/08; G01N 33/56966; A61K 39/00

(57) ABSTRACT

The present invention relates to compositions and method for targeting antigen presenting cells. In particular, the present invention relates to targeting peptides and methods of using the peptides to target molecules of interest to dendritic cells.

### FIELD OF THE INVENTION

The present invention relates to compositions and method for targeting antigen presenting cells. In particular, the present invention relates to targeting peptides and methods of using the peptides to target molecules of interest to dendritic cells.

### BACKGROUND OF THE INVENTION

Vaccination is amongst the most efficient forms of immunotherapy. Indeed, there has been significance decline in morbidity and mortality with most infectious diseases since the use of vaccines. Advanced knowledge in the molecular and cellular mechanisms underlying effective immune responses has revolutionized vaccine development over the past decades. Targeting antigens to dendritic cells (DCs) is a new concept aimed at enhancing immunity. The current targeting strategies focus mainly on distinct DC subsets and use antibodies. However, recent studies suggest that multiple DC subsets are required to induce optimal T cell immunity.

Thus, novel delivery technologies and further refinement of the existing methods are warranted. Additional targeting moieties and targeting a single receptor expressed by several antigen presenting cells are needed in the art.

### SUMMARY OF THE INVENTION

The present invention relates to compositions and method for targeting antigen presenting cells (e.g., dendritic cells (DC), macrophages, or monocytes). In particular, the present invention relates to targeting peptides and methods of using the peptides to target molecules of interest to dendritic cells.

Embodiments of the present invention provide a polypeptide comprising: an antigen presenting cell (APC) targeting peptide (e.g., optionally linked to a molecule of interest). The present invention is not limited to a particular APC targeting peptide. In some embodiments, the APC targeting peptide has the amino acid sequence X₍ₙ₎LPWLX(ₘ₎ (SEQ ID NO:13), wherein X is any amino acid, and m and n are integers. In some embodiments, the targeting peptide comprises the amino acid sequence NWXLXWLX(ₘ₎W (SEQ ID NO:29), where m is an integer from 2-6. In some embodiments, the leucines (L) in the aforementioned sequences are replaced by amino acids of similar properties (e.g., conservative substitutions such as valine (V) or isoleucine (I)). In some embodiments, the targeting peptide comprises the amino acid sequence NWYLPWLGTNDW (SEQ ID NO:17), or derivatives thereof (e.g., XWYLPWLGTNDW (SEQ ID NO:15), NWXLPWLGTNDW (SEQ ID NO:30), NWYLXWLGTNDW (SEQ ID NO:31), NWYLPWLXTNDW (SEQ ID NO:32), NWYLPWLGXNDW (SEQ ID NO:33), NWYLPWLGTXDW (SEQ ID NO:34), NWYLPWLGTNXW (SEQ ID NO:35), NWYLPWLGTNW (SEQ ID NO:36), NWYLPWLGTDW (SEQ ID NO:37), or NWYLPWLGTW (SEQ ID NO:38), wherein X denotes any amino acid). In some embodiments, the targeting peptide comprises the amino acid sequence NWYzPWLGTNDW (SEQ ID NO:39), NWYLPWzGTNDW (SEQ ID NO:40) or NWYzPWzGTNDW (SEQ ID NO:41), wherein z denotes an amino acid with a hydrophobic branched aliphatic side chain (e.g., L, V, I). In some embodiments, the APC targeting peptide has the amino acid sequence XWYLPWLG (SEQ ID NO:14) or XWYLPWLGTNDW (SEQ ID NO:15), wherein X is any amino acid, e.g., NWYLPWLG (SEQ ID NO:16) or NWYLPWLGTNDW (SEQ ID NO:17). In some embodiments, the molecule of interest is an antigen (e.g., a cancer antigen or a foreign antigen), and antisense compound, an aptamer, or an siRNA. In some embodiments, the polypeptide further comprises the sequence LTVSPWY (SEQ ID NO:18). In some embodiments, the APC targeting peptide and molecule of interest are in the same (e.g., a fusion polypeptide) or different (e.g., complexed or non-covalently linked) molecules. In some embodiments, the targeting peptide is coupled to a solid support (e.g., a bead (e.g., magnetic bead), column, etc.).

Further embodiments of the present invention provide a nucleic acid encoding the aforementioned polypeptides, vectors comprising the nucleic acids, or compositions comprising the nucleic acid. In some embodiments, the vector is a bacteriophage and a viral vector (e.g., that displays the polypeptide on a surface. In some embodiments, the composition further comprises an adjuvant and/or a pharmaceutically acceptable carrier. In some embodiments, the composition is a vaccine.

Certain embodiments provide compositions (e.g., pharmaceutical compositions), kits, articles of manufacture (e.g., solid supports) comprising the aforementioned peptides and their use in any of the methods described herein.

Additional embodiments of the present invention provide a method or use of inducing an immune response, comprising: administering any one of the aforementioned polypeptides, nucleic acids, vectors, or compositions to a subject, wherein the administering induces an immune response against the molecule of interest. In some embodiments, the immune response is a T-cell mediated immune response. In some embodiments, the immune response is against a cancer cell or a foreign antigen.

The present invention also provides a method of gene silencing in an APC, comprising: administering any one of the aforementioned polypeptides, nucleic acids, vectors, or compositions to a subject, wherein the administering results in gene silencing in the APC.

In further embodiments, the present invention provides a method for separation of cells with binding affinity for the aforementioned peptides, comprising: contacting a sample comprising cells (e.g., monocytes or dendritic cells) with a solid support comprising the peptides, and identifying cells that bind or are excluded from the peptide.

Additional embodiments are specifically contemplated, including those described herein.

### BRIEF DESCRIPTION OF THE DRAWINGS

FIG. 1 shows selection of DC-binding peptides. (A) Enrichment of DC-binding phages. (B) Representative examples of phage binding to iDCs.

FIG. 2 shows characterization of binding specificity. (A) Inhibition of the phage binding by synthetic peptides. (B) Binding of 6-IAF conjugated NW peptide to iDCs. (C) Fluorescence images. (D). The nuclei were visualized with Hoechst 33342 staining Data are representative of at least 4 independent experiments.

FIG. 3 shows that peptide binding did not affect the phenotype and function of DCs. Mature DCs were incubated (A) with or without (B) NW peptide (15 μM) for 48 h at 37° C. and then the expression of CD80, CD83, CD86, and HLA-DR molecules were analyzed by flow cytometry. (C) MLR assay.

FIG. 4 shows that NW-peptide can deliver small and large molecules to iDCs. (A) Biotinylated NW peptide or control peptide streptavidin-PE complexes were added to iDC and incubated for 60 min at 4° C. C) Analysis of NW phage binding to iDC by fluorescence microcopy. D) NW phage binding was analyzed by confocal microscopy.

FIG. 5 shows targeted delivery of CMV pp65 peptides. (A) Binding of 6IAF-conjugated peptides to iDCs. (B) Confocal microscopy images of iDC showing the binding of NW-60-mer fusion peptide after staining and incubation at 37° C. for 90 min.

FIG. 6 shows that targeted pp65 peptides to DCs enhanced T cell proliferation from CMV positive donors. (A) Semi mDCs were incubated with the indicated peptides at 4° C. for 60 min, washed to remove unbound peptides and then incubated at 37° C. for 60 min. Subsequently, they were added to autologous CD8 or CD4 T cells (10⁵ cells/well), cultured at 37° C. for 5 days and proliferation was monitored by [³H]-thymidine incorporation. (B) Dextramer staining of CTL against NLVPMVATV (SEQ ID NO:19) epitope. (C) The cells were also stained with HIV-1 pentamer.

FIG. 7 shows activation of naïve T cells by NW peptide-targeted delivery.

FIG. 8 shows NW peptide-targeted delivery to whole blood activated T cells. (A) PBMCs from HLA-A2+/CMV-positive donors were incubated with the indicated fusion peptides for 60 min at 4° C., washed and then incubated at 37° C. for 12 days. T-cell proliferation was monitored by thymidine incorporation. (B) Dextramer staining of CD8 T cells against NLVPMVATV (SEQ ID NO:19) epitope. (C) As in A and B, but monocyte-depleted PBMCs were used. (D) INF-γ and IL-10 levels in PBMC culture supernatants determined by ELISA.

FIG. 9 shows targeted vs spontaneous uptake of antigens by DCs.

FIG. 10 shows uptake and gene silencing by the NW-peptide siRNA conjugates. (A) Epifluorescence images of iDCs showing the binding of the peptide-siRNA conjugates. B) Confocal microscopy images showing the internalization of the peptide-siRNA conjugates. (C) Inhibition of galectin-3 gene expression by peptide siRNA conjugates.

FIG. 11 shows inhibition of the NW phage binding by the NW peptide and its mutant peptides. (A). Mean fluorescence intensities are show. B. A dose-dependent inhibition of the phage binding was obtained with the NW peptide (IC50=0.5 μM. Peptide concentrations 1, 2, 3 and 4, correspond to 0.4, 2, 8, and 20 μM.

FIG. 12 shows that asparagine 10 and aspartic acid are not required for the NW peptide binding. Mean fluorescence intensities are shown.

FIG. 13 shows effects of conservative amino acid replacements on peptide binding. A) Conservative replacement of tryptophan (W) by either tyrosine (Y) or phenylalanine (F) abolished the binding of the NW peptide to monocytes. B) Conservative replacement of leucine (L) with valine (V) did not affect the binding, replacement with isoleucine (I) partially inhibited the NW binding to monocytes. C) Mean fluorescence intensities are shown.

FIG. 14 shows that the NW peptide enhanced the delivery of Mart-1 peptide to blood APCs.

FIG. 15 shows depletion of monocytes from peripheral blood mononuclear cells.

### DEFINITIONS

A used herein, the term “immune response” refers to a response by the immune system of a subject. For example, immune responses include, but are not limited to, a detectable alteration (e.g., increase) in Toll receptor activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the term “immunity” refers to protection from disease (e.g., preventing or attenuating (e.g., suppression) of a sign, symptom or condition of the disease) upon exposure to a microorganism (e.g., pathogen) capable of causing the disease. Immunity can be innate (e.g., non-adaptive (e.g., non-acquired) immune responses that exist in the absence of a previous exposure to an antigen) and/or acquired (e.g., immune responses that are mediated by B and T cells following a previous exposure to antigen (e.g., that exhibit increased specificity and reactivity to the antigen)).

As used herein, the term “immunogen” refers to an agent (e.g., a microorganism (e.g., bacterium, virus or fungus) and/or portion or component thereof (e.g., a protein antigen)) that is capable of eliciting an immune response in a subject. In some embodiments, immunogens elicit immunity against the immunogen (e.g., microorganism (e.g., pathogen or a pathogen product)).

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

As used herein, the term “antisense compound” refers to an oligomeric compound that is at least partially complementary to a target nucleic acid molecule to which it hybridizes. In certain embodiments, an antisense compound modulates (increases or decreases) expression of a target nucleic acid. Antisense compounds include, but are not limited to, compounds that are oligonucleotides, oligonucleosides, oligonucleotide analogs, oligonucleotide mimetics, and chimeric combinations of these. Consequently, while all antisense compounds are oligomeric compounds, not all oligomeric compounds are antisense compounds.

As used herein, the term “antisense oligonucleotide” refers to an antisense compound that is an oligonucleotide. The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

As used herein, the term “aptamer” refers to nucleic acid (e.g., oligonucleotide) or peptide molecules that bind to a specific target molecule. In some embodiments, aptamers are created by selecting them from a large random sequence pool. However, aptamers can also be isolated from nature. In some embodiments, aptamers are used for basic research industrial and clinical purposes as macromolecular drugs.

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “sample” as used herein is used in its broadest sense. In one sense it can refer to a tissue sample. In another sense, it is meant to include a specimen or culture obtained from any source, as well as biological. Biological samples may be obtained from animals (including humans) and encompass fluids, solids, tissues, and gases. Biological samples include, but are not limited to blood products, such as plasma, serum and the like. These examples are not to be construed as limiting the sample types applicable to the present invention. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the term “peptide” refers to a polymer of two or more amino acids joined via peptide bonds or modified peptide bonds. As used herein, the term “dipeptides” refers to a polymer of two amino acids joined via a peptide or modified peptide bond.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified”, “mutant”, and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions with its various ligands and/or substrates.

As used herein, the term “purified” or “to purify” refers to the removal of contaminants from a sample. For example, antigens are purified by removal of contaminating proteins. The removal of contaminants results in an increase in the percent of antigen (e.g., antigen of the present invention) in the sample.

### DETAILED DESCRIPTION OF THE INVENTION

The present invention relates to compositions and method for targeting antigen presenting cells. In particular, the present invention relates to targeting peptides and methods of using the peptides to target molecules of interest to APCs.

Advanced knowledge in the molecular and cellular mechanisms underlying effective immune responses has revolutionized vaccine development over the past decades. Targeting antigens to dendritic cells (DCs) is a new concept aimed at enhancing immunity. Today, the most used strategy for DC vaccine is based on isolating monocytes from blood of patients and exposing them to differentiation/maturation stimuli. Subsequently, these monocyte-derived DCs are loaded with tumor antigens or mRNA and then re-injected into the patients. However, such ex-vivo generated DCs migrate poorly in-vivo and express immunosuppressive factors such as interleukin 10 and indoleamine 2,3-dioxygenase, thus affecting the efficacy of DC cancer vaccines. Moreover, the process used to create monocyte-derived DCs for vaccination is complex, expensive and cannot be applied for all patients. A more direct and less laborious strategy is to target tumor antigen to DCs in vivo-via DC surface receptors. Both in-vitro and in-vivo APC targeting reduces the antigen concentrations.

The identification of receptors that are more or less specifically expressed in DCs has resulted in the development of vaccination strategies that target DCs through the use of antibodies specific for these receptors. The current targeting strategies using antibodies focus mainly on distinct DC subsets. Moreover, large antigen-antibody conjugates may have disadvantages such as reduced tissue penetration. The use of mouse antibodies in humans is also expected to induce high immunogenicity although some humanized antibodies were developed. Thus, novel delivery technologies and further refinement of the existing methods are warranted. Additional targeting moieties and targeting a single receptor expressed by several antigen presenting cells are needed in the art.

Embodiments of the present disclosure provide APC targeting and binding peptides capable of binding to a receptor expressed by several antigen presenting cells such as monocytes-derived dendritic cells and blood myeloid dendritic cells. In contrast to antibodies, peptides represent important targeting tools because of their excellent tissue penetration and easy synthesis and conjugation to antigen or therapeutic molecules. The peptides find use in both clinical vaccine development and cancer immunotherapy.

Targeting antigens to DCs finds use in clinical vaccine development and cancer immunotherapy, where antigen delivery to DCs is important for the induction of naïve and memory immune responses. In the present study, we have identified DC-targeting peptides from peptide phage libraries. The targeting potential of the NW peptide was demonstrated in the context of phage, streptavidin protein, and pp65 peptides. Moreover, the NW peptide was able to facilitate siRNA delivery to DCs, thus offering co-delivery of antigens and toll-like receptor ligands such as CpG DNA oligonucleotides to DCs. In this respect, the data indicated that the NW-peptide can direct CpG oligonucleotides to DCs.

Antigen targeting to DCs is usually accomplished by coupling the antigens to antibodies specific for particular DC surface receptors such as CD205, mannose receptor, the β2 integrin CD11c, or the C-type lectin receptor Clec9A (Tacken et al. (2007) Nature Rev. Immunol. 7, 790-802). In some studies this strategy was successfully applied for efficient induction of T-cell responses. However, the development of additional targeting moieties that facilitate antigens and/or nucleic acids delivery to immune cells such as DCs is warranted as current targeting strategies are still far from being ideal. Consistent with it targeting potential, incubation of PBMCs from HLA-A2+/CMV positive with the NW-pp65 fusion peptide led to a significant increase in the proportion of CD8 and CD4 T cells that produced IFN-γ. Under the same conditions, untargeted pp65 peptide induced very low response. Moreover, targeting via the NW peptide activated naïve T cells from HLA-A2+/CMV negative donors. The ability to expand ex-vivo T cell precursors for potential antigens is useful not only for the generation of antigen-specific T cells for clinical applications but also for validating candidate antigens as immunogenic and analyzing the frequency T cell precursors in naïve repertoires.

In general, targeting exogenous antigens via specific receptors can drive the immune response either towards class II MHC-restricted CD4 T cell helper response or to class I MHC-restricted CD8 cytotoxic T cell response via cross-presentation, and therefore be an effective strategy for inducing anti-viral or anti-tumor immune responses (Kurts et al., (2010) Nat Rev Immunol. 2010, 403-414). In humans, antigen cross-presentation is promoted upon antigen uptake through DEC-205 and FcγR in-vitro as well as in-vivo (Bozzacco et al., (2007) PNAS, 104, 1289-94; Tsuji et al., (2010) J. Immunol. 186, 1218-27; Liu et al., (2006) J. Immunol. 177, 8440-7). Langerin (CD207)-targeted uptake induced both CD4 and CD8 T-cell responses (Regnault et al., (1999) J. Exp Med 189, 371-380). Similarly, the NW peptide receptor targeted delivery induces CD4 T-cell response, albeit significantly less to the CD8 T cell response. Enhancing cross-presentation is an effective way to improve cytotoxic CD8 T-cell responses against tumors; hence NW peptide-targeted delivery is useful for cancer immunotherapy.

Although the phage libraries were pre-absorbed on human monocytes, the NW peptide bound as strongly to monocytes as to dendritic cells (Table II). This indicates that the introduced subtraction step did not eliminate the phage displaying the NW peptide. While there is no evidence that monocytes are directly involved in antigen presentation and T-cell priming in-vivo, recent studies indicated that they play important roles in transporting antigens to the lymph nodes and as a source of inflammatory DCs (Auffray et al., (2009) Annu Rev. Immunol. 27, 669-92; Leiriao et al., (2012) Eur. J. Immunol. 42, 2042-2051). Although the NW peptide bound to human monocytes, a strong CD8 T-cell response was obtained using either whole PBMCs or monocyte-depleted PBMCs. Therefore, the targeting receptor does not need to be exclusively expressed by DCs. Other studies have also shown that efficient immune responses are still generated when other cell types as well as the DCs receive the targeted antibody-antigen complexes (Tacken et al., (2007) Nature Rev. Immunol. 7, 790-802; He et al., (2007) J. Immunol. 178, 6259-6267). Notably, the currently used targeting receptors are not specific for DCs (Tacken et al., supra). For example, DC-205 is expressed by DCs, but expression is also present on monocytes, B lymphocytes, NK cells and T lymphocytes (Kato et al., (2006) Int Immunol 18, 857-869). CD206 is expressed by DCs, monocytes, macrophages, and endothelial cells. The NW peptide, however, did not bind to T cells, B cells, NK cells and other tested human cells (Table II), indicating that its receptor is not expressed by these cells. The more efficient internalization, trafficking, and loading onto MHC class I and II pathways in DCs compared to monocytes, contributes to the NW peptide specificity in targeting DCs. In support of this, the NW-pp65 fusion peptides were more efficiently internalized by iDCs and mDCs compared to blood monocytes. Notably, the NW peptide bound efficiently to blood myeloid DCs and plasmacytoid DCs, thus underlying its targeting of bone marrow-derived DCs. Similar to monocyte-derived DCs, myeloid blood DC effectively internalized the NW-streptavidin complexes and phage particles.

Cytomegalovirus reactivation with progression to disease is a major cause of morbidity and mortality in immunocompromised recipients of bone marrow transplants (Meyers et al., (1986) Risk factors for cytomegalovirus infection after human marrow transplantation. J Infect Dis. 153, 478-488). Restoration of immune responses against CMV using CMV-specific T cells has shown promise in the treatment of CMV-associated disease in patients resistant to conventional viral therapies (Patel et al., (2012) Am J. Transplant, 12, 539-44). The CD8 T-cell response to CMV is dominated by the structural protein pp65, which is targeted by 70% to 90% of CMV-specific T cells (Wills et al., (1996) J Virol. 70, 7569-7579). The NW-pp65 peptides efficiently expanded pp65-specific T cells present in CMV positive donors. Most of the immuno-dominant peptides from pp65 protein that are restricted to specific HLA molecules are being identified, and these can also be fused to the NW peptide to induce CMV-specific cytotoxic CD8 T cells in the absence of live virus. Antigen targeting via the NW peptide is another potential activator of primary virus-specific T cells as well as diagnostic tool for detection of CMV cellular immunity in graft material before transplantation (Yao et al., (2008) Clin Infect Dis 46, 96-105). With respect to viral infection and cancer immunotherapy, some studies have shown that CD4 T cells are essential to sustain the CD8 responses, activate NK cells, macrophages, and B cells (Sant et al., (2012) J Exp Med. 209, 1391-5). Hence, the activation of CD4 and CD8 T cells by NW-peptide targeting to DCs has enormous clinical applications.

The development of agents capable of efficient delivery of siRNA to immune cells has been challenging. In terms of in-vitro transfection, primary cells are usually more difficult to transfect than immortalized cancer cells (Goffinet et al., (2006) FASEB J, 20, 500-502). The current study shows that the NW peptide is suitable for delivering siRNAs to primary dendritic cells.

### I. Targeting Peptides

Embodiments of the present invention provide APC targeting and binding peptides. Dendritic cells (DCs) are key regulators of T and B cell immunity, owing to their superior ability to capture, process and present antigens compared to other antigen-presenting cells (APCs). In fact, they are the only APCs capable of activating naïve T cells (Banchereau et al., (1998) Nature 392, 245-52). Given their role to link innate and adaptive immunity, a strong attention has been developed in their use in immunotherapies. Attempts to harness the ability of these cells to treat, for example, cancers have focused mainly on strategies involving the ex-vivo antigen loading of autologous monocyte-derived DCs that are re-administered to the patients (Palucka et al., (2010) Immunity 33, 464-478). However, such ex-vivo generated DCs, migrate poorly in-vivo and express immunosuppressive factors such as interleukin (IL)-10 and indoleamine 2,3-dioxygenase, thus affecting the efficacy of DC cancer vaccines (Flatekval et al., (2009) Immunology 128, e837-e848).

The identification of receptors that are more or less specifically expressed in DCs has resulted in the development of vaccination strategies that target DCs through the use antibodies specific for these receptors (Tacken et al., (2011) Sem Immunol. 23, 12-20). In general the antigens were either chemically coupled or genetically fused to antibodies, in order to direct them to DCs. Several of the currently used targeted receptors belong to the C-type lectin receptor family (Johnson et al., (2008) Clin Cancer Res 14, 8169-77; Wei et al., (2009) Clin Cancer Res 15, 4612-21; Kretz-Rommel et al., (2007) J Immunother 30, 715-26; Hangalapura et al., (2011) Cancer Res 71, 5827-5837; Birkholz et al., (2010) Blood, 116, 2277-2285; Serre et al., (1998) J Immunol 161, 6059-67; He et al., (2007) J. Immunol. 178: 6259-6267). Among these are endocytic receptor DEC205 (CD205), the mannose receptor C type 1 (DC206), and intercellular adhesion molecule 3 (ICAM3) (Birkholz et al., supra; Tacken et al., (2005) Blood 106, 1278-1285). Although strong T-cell responses have been achieved, antibody targeting may provide additional activation signals that may negatively affect T-cell activation. Furthermore, large antigen-antibody conjugates may have disadvantages such as reduced tissue penetration. The use of mouse antibodies in humans is also expected to induce high immunogenicity although some humanized antibodies were developed (Tacken et al., (2005) Blood 106, 1278-1285). In view of these potential challenges, the discovery of additional targeting moieties is warranted. Moreover, it is important to extend the spectrum of DC targeting receptors that can facilitate cross-presentation of exogenous antigens, a necessary step for the induction of cytotoxic CD8 T lymphocytes against tumors and viruses (Kurts et al., (2010) Nat Rev Immunol. 2010, 403-414).

An alternative approach for antigen delivery is the use of short peptides targeted to specific DC receptors. In contrast to large molecules, peptides would represent important targeting tools because of their excellent tissue penetration and easy synthesis and conjugation to antigens (Shadidi, M., and Sioud, M. (2003) Drug Resist. Update 6, 363-71). During the last years, peptide phage libraries have provided a new opportunity to identify peptides with desired binding specificity and/or function (Smith, G. P., and Scott, J. K. (1993) Methods Enzymol. 217, 228-257; Laakkonen et al., (2002) Nat Med, 8, 751-755; Costantini et al. (2012) Peptides 38: 94-99). Another advantage of this technology is that the selection of cell-binding peptides is not only based on the expression profile of the receptor but also to its accessibility to extracellular interactions.

Peptide phage libraries were used either to probe the specificities of patient serum antibodies or to select cancer cell-binding peptides (Dybwad et al. (1993) Eur. J. Immunol. 23, 3189-3193; Hansen et al., (2001) Mol. Med 7: 230-239; Shadidi, M., and Sioud, M. (2003) FASEB J 17, 256-8). A variety of molecules fused to one of the selected peptides (LTVSPWY) has been designed to target cancer cells in-vitro and in-vivo (Shadidi, M., and Sioud, M. (2003) FASEB J 17, 256-8; Wang et al., (2007) Cancer Res 67, 3337-44; Luo et al., (2011) FASEB J 25, 1-9). In experiments described herein, biopanning of peptide phage libraries on monocyte-derived immature DCs (iDCs) and binding peptides were selected. One of the selected peptides (NW-peptide) bound with high affinity to DCs and was able to direct proteins and small interfering RNAs (siRNAs) to DCs. Moreover, NW-peptide targeting of long peptides from CMV-pp65 protein to DCs enhanced memory and naïve T-cell responses.

The present disclosure is not limited to a particular APC targeting peptide. In some embodiments, the APC targeting peptide has the amino acid sequence X₍ₙ₎LPWLX(ₘ₎ (SEQ ID NO:13), wherein X is any amino acid, and m and n are integers. For example, in some embodiments, the APC targeting peptide has the amino acid sequence XWYLPWLG (SEQ ID NO:14) or XWYLPWLGTNDW (SEQ ID NO:15), wherein X is any amino acid, NWYLPWLG (SEQ ID NO:16) or NWYLPWLGTNDW (SEQ ID NO:17). In some embodiments, the targeting peptide is NWYLPWLGTNDWC (SEQ ID NO:1). In some embodiments, the targeting peptide is a variant, homolog, or modified version of SEQ ID NO:1.

In some embodiments, the targeting peptide comprises the amino acid sequence NWYLPWLGTNDW (SEQ ID NO:17), or derivatives thereof (e.g., XWYLPWLGTNDW (SEQ ID NO:15), NWXLPWLGTNDW (SEQ ID NO:30), NWYLXWLGTNDW (SEQ ID NO:31), NWYLPWLXTNDW (SEQ ID NO:32), NWYLPWLGXNDW (SEQ ID NO:33), NWYLPWLGTXDW (SEQ ID NO:34), NWYLPWLGTNXW (SEQ ID NO:35), NWYLPWLGTNW (SEQ ID NO:36), NWYLPWLGTDW (SEQ ID NO:37), or NWYLPWLGTW (SEQ ID NO:38), wherein X denotes any amino acid). In some embodiments, the targeting peptide comprises the amino acid sequence NWYzPWLGTNDW (SEQ ID NO:39), NWYLPWzGTNDW (SEQ ID NO:40) or NWYzPWzGTNDW (SEQ ID NO:41), wherein z denotes an amino acid with a hydrophobic branched aliphatic side chain (e.g., L, V, I).

In the present context, a homologous sequence is taken to include an amino acid sequence which may be at least 80%, at least 85%, at least 90%, at least 95%, at least 96%, at least 97%, at least 98% or at least 99%, identical to the subject sequence. Typically, the homologs will comprise the same active sites and other functional sequences as the subject amino acid sequence. Although homology can also be considered in terms of similarity (e.g., amino acid residues having similar chemical properties/functions), in the context of the present invention it is preferred to express homology in terms of sequence identity.

Sequence identity comparisons can be conducted by eye, or more usually, with the aid of readily available sequence comparison programs. These commercially available computer programs use complex comparison algorithms to align two or more sequences that best reflect the evolutionary events that might have led to the difference(s) between the two or more sequences. Therefore, these algorithms operate with a scoring system rewarding alignment of identical or similar amino acids and penalizing the insertion of gaps, gap extensions and alignment of non-similar amino acids. The scoring system of the comparison algorithms include:
i) assignment of a penalty score each time a gap is inserted (gap penalty score),
ii) assignment of a penalty score each time an existing gap is extended with an extra position (extension penalty score),
iii) assignment of high scores upon alignment of identical amino acids, and
iv) assignment of variable scores upon alignment of non-identical amino acids.
   Most alignment programs allow the gap penalties to be modified. However, it is preferred to use the default values when using such software for sequence comparisons.

The scores given for alignment of non-identical amino acids are assigned according to a scoring matrix also called a substitution matrix. The scores provided in such substitution matrices are reflecting the fact that the likelihood of one amino acid being substituted with another during evolution varies and depends on the physical/chemical nature of the amino acid to be substituted. For example, the likelihood of a polar amino acid being substituted with another polar amino acid is higher compared to being substituted with a hydrophobic amino acid. Therefore, the scoring matrix will assign the highest score for identical amino acids, lower score for non-identical but similar amino acids and even lower score for non-identical non-similar amino acids. The most frequently used scoring matrices are the PAM matrices (Dayhoff et al. (1978), Jones et al. (1992)), the BLOSUM matrices (Henikoff and Henikoff (1992)) and the Gonnet matrix (Gonnet et al. (1992)).

Suitable computer programs for carrying out such an alignment include, but are not limited to, Vector NTI (Invitrogen Corp.) and the ClustalV, ClustalW and ClustalW2 programs (Higgins D G & Sharp P M (1988), Higgins et al. (1992), Thompson et al. (1994), Larkin et al. (2007). A selection of different alignment tools is available from the ExPASy Proteomics server. Another example of software that can perform sequence alignment is BLAST (Basic Local Alignment Search Tool), which is available from the webpage of National Center for Biotechnology Information (Altschul et al. (1990) J. Mol. Biol. 215; 403-410).

Once the software has produced an alignment, it is possible to calculate % similarity and % sequence identity. The software typically does this as part of the sequence comparison and generates a numerical result.

In one embodiment, it is preferred to use the ClustalW software for performing sequence alignments. Preferably, alignment with ClustalW is performed with the following parameters for pairwise alignment:

| | |
|---|---|
| Substitution matrix: | Gonnet 250 |
| Gap open penalty: | 20 |
| Gap extension penalty: | 0.2 |
| Gap end penalty: | None |

ClustalW2 is for example made available on the internet by the European Bioinformatics Institute at the EMBL-EBI webpage under tools—sequence analysis—ClustalW2.

In another embodiment, it is preferred to use the program Align X in Vector NTI (Invitrogen) for performing sequence alignments. In one embodiment, Exp10 has been may be used with default settings:

Gap opening penalty: 10
Gap extension penalty: 0.05
Gap separation penalty range: 8
Score matrix: blosum62mt2

The sequences, particularly those of variants, homologues and derivatives of SEQ ID NO:1 may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

The present invention also encompasses conservative substitution (substitution and replacement are both used herein to mean the interchange of an existing amino acid residue, with an alternative residue) that may occur, e.g. like-for-like substitution such as basic for basic, acidic for acidic, polar for polar etc. Non-conservative substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyridylalanine, thienylalanine, naphthylalanine and phenylglycine.

Conservative substitutions that may be made are, for example within the groups of basic amino acids (Arginine, Lysine and Histidine), acidic amino acids (glutamic acid and aspartic acid), aliphatic amino acids (Alanine, Valine, Leucine, Isoleucine), polar amino acids (Glutamine, Asparagine, Serine, Threonine), aromatic amino acids (Phenylalanine, Tryptophan and Tyrosine), hydroxyl amino acids (Serine, Threonine), large amino acids (Phenylalanine and Tryptophan) and small amino acids (Glycine, Alanine).

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid, 7-amino heptanoic acid*, L-methionine sulfone^{#*}, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-conservative substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al. (1992), Horwell D C. (1995).

In some embodiments, targeting peptides comprise a linker for fusing the targeting peptide to a polypeptide or nucleic acid of interest. In some embodiments, targeting peptides comprise a label or other detectable moiety (e.g., for use in diagnostic applications). In some embodiments, targeting peptides comprise a linker for fusing the targeting peptide to a polypeptide or nucleic acids of interest. In further embodiments, linkers such as GGGS (SEQ ID NO:20) or GGGSRRR (SEQ ID NO:21) are utilized to increase peptide solubility in water.

The present disclosure is not limited to a particular molecule of interest. Examples include, but are not limited to antigens, antisense molecules, siRNA, and aptamers.

The present disclosure also relates to an article (e.g., solid support), composition (e.g. pharmaceutical composition), or kit comprising APC (e.g., those described herein) targeting peptides or fusion proteins thereof for diagnostic, medical or scientific purposes.

### II. Uses

Embodiments of the present invention relate to peptide compositions, vaccine compositions, therapeutic compositions, kits and uses thereof. Such compositions direct therapeutic molecules such cancer vaccines and small interfering RNAs (siRNAs) to dendritic cells and/or monocytes, leading to effective cellular and/or humoral immunity. Such delivery strategies find use in the specific delivery of a wide variety of vaccine antigens (e.g. tumor antigens, viral antigens, bacterial antigens) to antigen presenting cells (APC). The present invention is not limited to delivery vaccines. Any therapeutic molecule can be directed to APC through the peptides described herein. Furthermore, the vaccine antigens can be co-expressed on the cell surface of bacteriophage/virus along with a targeting peptide.

The targeting peptides described herein find use in a variety of application. Examples include, but are not limited to, cancer immunotherapy, vaccine delivery, gene silencing, cell purification and separation, and diagnostic applications.

The targeting peptides find use in the delivery of any number of molecules of interest (e.g., antigens) to APCs. In some embodiments, antigens are peptide antigens. In other embodiments, antigens are nucleic acids. In some embodiments, molecules of interest are siRNAs for use in gene silencing applications.

A. Vaccines

The targeting peptides according to embodiments of the present invention may be suitable for induction of an immune response against any polypeptide of any origin. Any antigenic sequence of sufficient length that includes a specific epitope may be used as the antigenic unit in the proteins according to the invention. Accordingly in some embodiments, the antigenic unit comprises an amino acid sequence of at least 9 amino acids corresponding to at least about 27 nucleotides in a nucleic acids sequence encoding such antigenic unit. Such an antigenic sequence may be derived from cancer proteins or infectious agents. Examples of such cancer sequences are telomerase, more specifically hTERT, tyrosinase, TRP-1/TRP-2 melanoma antigen, prostate specific antigen and idiotypes. The infectious agents can be of bacterial, e.g. tuberculosis antigens and OMP31 from brucellosis, or viral origin, more specifically HIV derived sequences like e.g. gp120 derived sequences, glycoprotein D from HSV-2, and influenza virus antigens like hemagglutinin, nuceloprotein and M2. Insertion of such sequences in a fusion with a targeting peptide of embodiments of the present invention can also lead to activation of both arms of the immune response. Alternatively the antigenic unit may be antibodies or fragments thereof, such as the C-terminal scFv derived from the monoclonal Ig produced by myeloma or lymphoma cells, also called the myeloma/lymphoma M component in patients with B cell lymphoma or multiple myeloma.

Compositions comprising a targeting peptide described herein fused or conjugated to a molecule of interest may be utilized for immunization of a subject, for example, by intramuscular or intradermal injection with or without a following electroporation.

The various units of fusion proteins according to the present invention may be operably linked via standard molecular biology methods, and the DNA transfected into a suitable host cell, such as NS0 cells, 293E cells, CHO cells or COS-7 cells. The transfectants produce and secrete the recombinant proteins.

Where appropriate, vaccine compositions additionally comprise a pharmaceutically compatible carrier. Suitable carriers and the formulation of such pharmaceuticals are known to a person skilled in the art. Suitable carriers are, e.g., phosphate-buffered common salt solutions, water, emulsions, e.g. oil/water emulsions, wetting agents, sterile solutions, etc. The pharmaceuticals may be administered orally or parenterally. The methods of parenteral administration comprise the topical, intra-arterial, intramuscular, subcutaneous, intramedullary, intrathekal, intraventricular, intravenous, intraperitoneal, or intranasal administration. The suitable dose is determined by the attending physician and depends on different factors, e.g. the patient's age, sex and weight, the kind of administration etc.

Indeed, a vaccine composition of the present disclosure may comprise one or more different agents in addition to the APC targeting molecule fused to an antigen. These agents or cofactors include, but are not limited to, adjuvants, surfactants, additives, buffers, solubilizers, chelators, oils, salts, therapeutic agents, drugs, bioactive agents, antibacterials, and antimicrobial agents (e.g., antibiotics, antivirals, etc.). In some embodiments, a vaccine composition comprises an agent or co-factor that enhances the ability of the antigenic unit to induce an immune response (e.g., an adjuvant). In some preferred embodiments, the presence of one or more co-factors or agents reduces the amount of antigenic unit required for induction of an immune response (e.g., a protective immune response (e.g., protective immunization)). In some embodiments, the presence of one or more co-factors or agents is used to skew the immune response towards a cellular (e.g., T-cell mediated) or humoral (e.g., antibody-mediated) immune response. The present invention is not limited by the type of co-factor or agent used in a therapeutic agent of the present invention.

Adjuvants are described in general in Vaccine Design—the Subunit and Adjuvant Approach, edited by Powell and Newman, Plenum Press, New York, 1995, incorporated by reference herein in its entirety for all purposes. The present invention is not limited by the type of adjuvant utilized (e.g., for use in a composition (e.g., a pharmaceutical composition)). For example, in some embodiments, suitable adjuvants include an aluminium salt such as aluminium hydroxide gel (e.g., alum) or aluminium phosphate. In some embodiments, an adjuvant may be a salt of calcium, iron, or zinc, or it may be an insoluble suspension of acylated tyrosine, or acylated sugars, cationically or anionically derivatized polysaccharides, or polyphosphazenes.

In general, an immune response is generated to an antigen through the interaction of the antigen with the cells of the immune system. Immune responses may be broadly categorized into two categories: humoral and cell-mediated immune responses (e.g., traditionally characterized by antibody and cellular effector mechanisms of protection, respectively). These categories of response have been termed Th1-type responses (cell-mediated response), and Th2-type immune responses (humoral response).

Stimulation of an immune response can result from a direct or indirect response of a cell or component of the immune system to an intervention (e.g., exposure to an antigenic unit). Immune responses can be measured in many ways including activation, proliferation, or differentiation of cells of the immune system (e.g., B cells, T cells, dendritic cells, APCs, macrophages, NK cells, NKT cells etc.); up-regulated or down-regulated expression of markers and cytokines; stimulation of IgA, IgM, or IgG titer; splenomegaly (including increased spleen cellularity); hyperplasia and mixed cellular infiltrates in various organs. Other responses, cells, and components of the immune system that can be assessed with respect to immune stimulation are known in the art.

B. Gene Silencing

In some embodiments, the targeting peptides described herein delivery siRNAs to dendritic cells for gene silencing application via RNAi. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments. In some embodiments, RNAi oligonucleotides are designed to target the junction region of fusion proteins.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Comers, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol Biol. 2005 May 13; 348(4):883-93, J Mol Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

In some embodiments, targeting molecules are used to target antisense oligonucleotides to APCs. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of target genes that the antisense oligonucleotide hybridizes to. In the context of the present invention, “modulation” means either an increase (stimulation) or a decrease (inhibition) in the expression of a gene.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a cancer marker of the present invention. The targeting process also includes determination of a site or sites within this gene for the antisense interaction to occur such that the desired effect, e.g., detection or modulation of expression of the protein, will result.

The present invention also includes pharmaceutical compositions and formulations that include compositions described herein. The present invention further relates to a pharmaceutical comprising the above described recombinant based proteins, DNA/RNA sequences, or expression vectors according to the invention. Where appropriate, this pharmaceutical additionally comprises a pharmaceutically compatible carrier. Suitable carriers and the formulation of such pharmaceuticals are known to a person skilled in the art. Suitable carriers are, for example, phosphate-buffered common salt solutions, water, emulsions, e.g. oil/water emulsions, wetting agents, sterile solutions etc. The pharmaceuticals may be administered orally or parenterally. The methods of parenteral administration comprise the topical, intra-arterial, intramuscular, subcutaneous, intramedullary, intrathekal, intraventricular, intravenous, intraperitoneal or intranasal administration. The suitable dose is determined by the attending physician and depends on different factors, e.g. the patient's age, sex and weight, the kind of administration etc.

C. Cell Binding and Purification

In some embodiments, the APC targeting peptides described herein find use in the identification of cells that bind to the peptides. In some embodiments, APC targeting peptides are affixed to a solid support (e.g., column, bead, etc.) and contacted with a sample. In some embodiments, cells (e.g., dendritic cells or monocytes) that bind to or are excluded from binding the peptide are identified and/or purified.

### EXPERIMENTAL

The following examples are provided in order to demonstrate and further illustrate certain preferred embodiments and aspects of the present invention and are not to be construed as limiting the scope thereof.

### Example 1

### Materials and Methods

### Cell Isolation.

Peripheral blood mononuclear cells (PBMCs) were obtained from buffy coats of healthy individuals and isolated by density gradient centrifugation (Lymphoprep, Nycomed Pharm, Oslo). Monocytes were prepared using plastic adherence. In brief, PBMCs were re-suspended in complete RPMI medium and allowed to adhere for 1 h at 37° C. Non-adherent cells were gently removed, washed and cryopreserved. The adherent cells were gently collected and then differentiated to iDCs by adding IL-4 (100 ng/ml) and GM-CSF (50 ng/ml) in complete RPMI medium for 5-6 days. TNF-α (100 ng/ml) was added to iDCs for 2 additional days in order to differentiate them into mature DCs (mDCs) that are characterized by high expression of CD83, CD80, and CD86 molecules. In some experiments, iDCs were incubated with TNF-α for only one day. These semi mDCs were used for T-cell activation in-vitro using targeted pp65 fusion peptides because they still have an activate antigen processing machinery when compared to mDCs. T-cell activation was performed in X-vivo 15 medium (Cambrex, Wiesbaden, Germany). Buffy coats from either HLA-A2/CMV-positive or negative donors were also obtained, and PBMCs/monocytes were prepared as indicated above. CD4 and CD8 T cells were isolated from non-adherent cells using specific bead-conjugated antibodies (Dynal Invitrogen, Oslo, Norway), washed and cryopreserved until use. In some experiments, CD14 monocytes were depleted from PBMCs using anti-CD14-conjugated magnetic beads (Dynal Invitrogen, Oslo, Norway). Memory T cells and T regulatory cells (Tregs) were depleted from PBMCs using anti-CD45RO-conjugated magnetic beads on MACS LD columns (Miltenyi Biotec GmbH, Gladbach, Germany). Blood CD1c+(BDCA-1) myeloid and CD303+(BDCA-2) plasmacytoid DC cells were labeled with the correspondent antibody-conjugated magnetic beads and purified by autoMACS Pro Separator instrument as described by the manufacturer's instructions (Miltenyi Biotec GmbH, Gladbach, Germany).

### Selection of DC-Binding Phages.

The PhD peptide phage libraries (7-mer and 12-mer) were purchased from New England Biolabs (Ipswich, Mass., USA). The phage libraries were amplified and tittered according to the manufacturer's instructions. Prior to biopanning on iDC, the libraries (10¹¹ TU each) were pre-absorbed on human monocytes for 1 h at room temperature (RT). Pre-absorbed libraries were added to iDC cultured in a T-25 tissue culture flask and then incubated for 2 h at room temperature with gentle agitation. Subsequently, the cells were incubated at 37° C. for 45 min in order to mediate phage internalization. The cells were washed 4 times with PBS pH 7.4 and twice with PBS pH 6.5 to remove unbound phages. Cell-associated phages were recovered by lysing the cells in 50-100 μl water, after which 500 μl elution buffer (0.1 M glycine-HCl pH 2.2, 1 mg/ml BSA) was added and the mixture was incubated for 30 min at RT followed by centrifugation for 5 min at 12000 rpm. The supernatant was collected and neutralized with ⅛ volume of 1M Tris-HCl pH 9.2. Phages were amplified in Escherichia Coli ER2537 and precipitated with ⅙ volume of 20% polyethylene glycol (PEG) 8000/2.5 M NaCl as described by the manufacturer's instructions. After 4 rounds of biopanning, amplified phages from all rounds were tested for binding. Moreover, single phage clones from the fourth round were amplified and tested for binding to iDCs using flow cytometry. The titer of each phage preparation was determined by plaque assay according to the manufacturer's instructions.

### DNA Sequencing.

DNA from individual positive phage clones were isolated using single-stranded M13 DNA isolation kit (Qiagen Norge, Oslo, Norway). The sequences of the phage-displayed peptides were deduced after sequencing the unique nucleotide region of the pIII protein using M13 sequencing primers (Eurofins MWG, Ebersberg, Germany).

### Phage Biotinylation.

Sulfo-NHS-biotin (Santa Cruz Biotechnology, Heidelberg, Germany) was dissolved in DMSO at 10 mg/ml and around 3 μl was added to the phage sample (10¹² TU/200 μl) and the mixture incubated for 2 h at RT with gentle shaking Subsequently, the volume was adjusted to 500 μl and phage particles were PEG-precipitated twice in order to remove free biotin.

### Analysis of Phage Binding to DCs by Flow Cytometry.

In brief, aliquots of DCs (10⁵) were divided into conical 96-well micro-plate, washed with PBS buffer containing 1% FCS, and then incubated with the amplified phages (≈10⁹ TU) for 30-60 min on ice. After washing, cells were incubated with biotinylated anti-M13 monoclonal antibody (Abcam) and then with phycoerythrin (PE)-conjugated streptavidin. Competition assays were performed by pre-incubating DCs with different amounts of the peptides for 15 min on ice. Then NW phage (≈10⁹ TU) was added and samples incubated for additional 60 min on ice. After washing, bound phages were detected as indicated above. Samples were analyzed by FACSCanto II flow cytometry.

### Peptides.

Peptides were synthesized by GeneCust Europe (Dudelange, Luxembourg). A cysteine residue (bolded letter) was added to the sequences to allow conjugation to thiol group-containing reagents such as 6-iodacetamidofluorescein (Invitrogen Dynal AS, Oslo, Norway) and to track the peptides by flow cytometry, epifluorescence and confocal microscopy. A biotin residue was added to the C-terminal of some peptides to allow the peptide binding to streptavidin-PE. All peptides were made by use of solid phase chemistry, purified to homogeneity (>85%) by reverse phase high-pressure liquid chromatography, and assessed by mass spectrometry. Letters in italics correspond to the CMV pp65 peptides conjugated to either the control or NW peptide.

(SEQ ID NO: 1)

| | |
|---|---|
| 1. | NWYLPWLGTNDWC (NW peptide) |

(SEQ ID NO: 2)

| | |
|---|---|
| 2. | NWYLPWLGTNDWGGGSC (NW peptide with G-linker) |

(SEQ ID NO: 3)

| | |
|---|---|
| 3. | NWYLPWLGTNDWGGGK-Biotin (NW-Biotin peptide) |

(SEQ ID NO: 4)

| | |
|---|---|
| 4. | NWYGAGAGTNDW (NW-mutant peptide) |

(SEQ ID NO: 5)

| | |
|---|---|
| 5. | GALDTTHHRPWTC (Control peptide) |

(SEQ ID NO: 6)

| | |
|---|---|
| 6. | GAGAAGGAGGGG (Control peptide) |

(SEQ ID NO: 7)

| | |
|---|---|
| 7. | GALDTTHHRPWTGGGK-Biotin (Control biotin pep- |
| | tide) |

(SEQ ID NO: 8)

| | |
|---|---|
| 8. | NWYLPWLGTNDWAGILARNLVPMVATVQGQNLC (NW-33-mer) |

(SEQ ID NO: 9)

| | |
|---|---|
| 9. | GAGAAGGAGGGGAGILARNLVPMVATVQGQNLC (GA-33-mer) |

(SEQ ID NO: 10)

| | |
|---|---|
| 10. | NWYLPWLGTNDWGCFTWPPWQAGILARNLVPMVATVQGQNLKYQEF |
| | FWDANDIYRIFAEL (NW-60-mer) |

(SEQ ID NO: 11)

| | |
|---|---|
| 11. | GALDTTHHRPWTGCFTWPPWQAGILARNLVPMVATVQGQNLKYQEF |
| | FWDANDIYRIFAEL (GA-60-mer). |

### Peptide Conjugation.

The conjugation of the peptides to either 6-iodacetamidofluorescein or siRNA was performed as described previously (Sioud, M., and Mobergslien, A. (2012) Bioconjugate Chem 23, 1040-1049). The disulfide linkage was formed between a thiol group of C terminal cysteine residue of the peptide and a 5′-thiol functionalized siRNA sense strand. The sequence of galectin 3 (Gal-3) siRNA sense strand is the following: 5′-GCUCCAUGAUGCGUUAUCU-3′ (SEQ ID NO:12). Modified siRNA duplexes with 5′-thiol sense strand were made and HPLC purified by Eurogentec (Seraing, Belgium).

### Autologous Stimulation of T Lymphocytes by DCs Exposed to Targeted Pp65 Peptides.

Peptides at a concentration of 5 μg/ml were incubated with semi mDCs from HLA-A2/CMV-positive donors for 60 min at 4° C. Subsequently, they were washed to remove unbound peptides. Autologous stimulation was done in 96-well tissue culture plates in X-vivo 15 medium. Briefly, DCs were mixed with 10⁵ autologous CD4 or CD8 T cells at a DC/lymphocytes ratios of 1/5 and 1/10 in a final volume of 250 μl. Cells were incubated for 5 days and then they were pulsed with [³H] thymidine and harvested 16 h later. [³H] thymidine incorporation was measured in a β-scintillation counter. In some experiments, the cells were cultured for 8 days and then stained with dextramers specific for pp65 NLVPMVATV (SEQ ID NO:19) epitope.

Autologous Stimulation of PBMCs with Pp65 Targeted Peptides.

PBMC from HLA-A2/CMV+ donors were thawed, washed and then incubated with the peptides (5 μg/ml) for 60 min at 4° C. Subsequently, the cells were washed twice and then plated at 2×10⁵ cells per well in 96-well tissue culture plate. Cells were cultured for 5 days and subsequently they were pulsed with [³H] thymidine and harvested 16 h later. In some experiments, the cells were cultured for 12 days and then stained with dextramers specific for pp65 NLVPMVATV (SEQ ID NO:19) epitope. Monocyte-depleted PBMCs were also used. To analyze primary immune responses against pp65 protein, PBMCs from HLA-A2+/CMV-negative donors were used. Autologous DCs were incubated with either NW-pp65 or GA-pp65 peptide (5 μg/ml) at 4° C. for 1 h, followed by three washes to remove unbound peptides. Subsequently, the cells were incubated with CD45RO-depleted PBMC (responder cells) and co-cultured for 10 days at ratio 1/10 (DC to responder cells) in X-vivo 15 medium supplemented with 10 ng/ml human IL-7 for 10 days. After 2 rounds of stimulation (8 days each) with autologous DC loaded with peptides, the cells were stained with dextramers specific for pp65 NLVPMVATV (SEQ ID NO:19) epitope. To test spontaneous uptake of peptides by DCs, PBMCs from HLA-A2+/CMV positive donors were incubated at 37° C. with various concentrations of untargeted or targeted pp65 peptide for 90 min. Subsequently, the cells were washed to remove unbound peptides and cultured at 37° C. for 12 days and then stained with dextramers specific for pp65 NLVPMVATV (SEQ ID NO:19) epitope.

### Dextramer Analysis of CMV Specific CD8 T Cells.

Dextramers with CMV (NLVPMVATV) (SEQ ID NO:19) and HIV (ILKEPVHGV) (SEQ ID NO:22) HLA-A2 specific antigens were obtained from Immudex (Copenhagen, Denmark). Antibodies against human CD8, CD19 and CD56 were obtained from eBioscience (San Diego, Calif., USA). Briefly, around 10⁶ cells were washed and resuspended in 50 μl staining buffer SB (PBS with 0.1% human serum albumin and 0.1% NaN₃) containing 1 mg/ml aggregated α-globulin and then stained with dextramer for 10 min in the dark at room temperature. Subsequently, antibodies against CD8, CD19 and CD56 were added and incubation continued for additional 20 min. Cells were washed and resuspended in SB containing 1% paraformaldehyd and analyzed on a BD SLR II flow cytometer. The data were analyzed using FCS Express (De Novo Software, Los Angeles, Calif., USA).

### Phenotypic Analysis of DCs:

Phenotype of DCs was analyzed by direct immunofluorescence staining of cell surface antigens using FITC or PE conjugated antibodies against CD80, CD83, CD86, HLA-DR, CCR7, CD40, and isotype controls. All antibodies were purchased from Dako (Glostrup, Denmark) or BD Biosciences (San Diego, Calif., USA). After staining on ice for 30 min, samples were washed twice and then analyzed by FACSCantoII flow cytometry

### Analysis of Peptide Binding by Flow Cytometry.

The cells were seeded onto 24-well plate (3×10⁵/well/0.5 ml) in X-vivo 15 medium and incubated overnight at 37° C. Subsequently, they were incubated with 6IAF-conjugated peptides (5 μg/ml) for 30 min at 37° C., gently scraped, washed 3 times and then analyzed by flow cytometry. Binding was also performed at 4° C.

### Analysis of Peptide Binding by Fluorescence Microscopy.

DCs were cultured in Lab-Tek chamber slides (Nalge Nunc International, Naperville, USA) for 24 h in X-vivo 15 medium. Then the medium was replaced with fresh medium and the cells were incubated with the peptides (5 μg/ml) for 60 min at 37° C. followed by 5 min incubation with Hoechst 33342 (Invitrogen Dynal AS, Oslo, Norway). The cells were washed twice with PBS and fixed with 4% paraformaldehyde for 15 min at 4° C. After washing, slides were covered with Dako cytomation fluorescent mounting medium and then images were taken with either epifluorescence (Leica DM RHC, Leica Microscopy As, Oslo, Norway) or confocal microscopy (Zeiss LSM 510, Olympus, Tokyo, Japan).

### Uptake of the NW-Peptide Streptavidin-PE Complexes by DCs.

Commercially available streptavidin-PE (1/200) was incubated with biotinylated NW peptide or control peptide (1 μg/ml) for 30 min at RT. Then the mixtures were added to DCs growing in Lab-Tek chamber slides (Nalge Nunc International, Naperville, USA) and incubated for 60 min at 4° C. The cells were washed 3 times with medium and incubated at 37° C. for 90 min to allow internalization of bound streptavidin-PE. To visualize the nuclei, Hoechst 33342 (Invitrogen Dynal AS, Oslo, Norway) was added to the cells for 5 min. Subsequently, the cells were fixed with 4% paraformaldehyde, washed and slides were covered with Dako cytomation fluorescent mounting medium followed by epifluorescence microscopy analysis. Confocal images were taken with an AxioVert 200 microscope (Carl Zeiss, Jena, Germany).

### Fluorescence Microscopy Analysis of Phage Binding.

DCs were incubated with biotinylated phages at 4° C. for 30 min. After washing, the cells were incubated with streptavidin-PE. Stained cells were re-suspended in 300 μl X-vivo 15 medium and cultured in Lab-Tek chamber slides at 37° C. for 90 min to allow internalization of bound phages. Hoechst 33342 dye was added to the cells for 5 min and then the cells were washed, fixed with 4% paraformaldehyde, and covered with Dako cytomation fluorescent mounting medium and analyzed with epifluorescence microscopy. Confocal images were obtained using an AxioVert 200 microscope (Carl Zeiss, Jena, Germany).

### Gene Silencing:

Immature DCs were seeded in a 6-well plate at a density of 10⁶ cells per well and incubated for 24 h prior to transfection. Then the medium was replaced by fresh X-vivo 15 medium (2 ml/well) containing peptide-siRNA conjugates or free siRNAs. Cells were harvested 48 h after addition of the test molecules and monitored for gene expression by Western blots.

### Western Blot Analysis:

Cells were resuspended in protein extraction buffer (PBS+1% NP-40) supplemented with protease inhibitor cocktail (Sigma-Aldrich Norge, Oslo, Norway) and incubated for 30 min on ice. After centrifugation, the supernatant were collected and protein contents were determined using Bio-Rad protein assay. Equal amounts of protein were resolved by electrophoresis on a 10% sodium dodecyl sulfate (SDS)-polyacrylamide gel and electrotransferred to nitrocellulose membrane. After blocking in 5% milk in TBS-Tween (1%) for 60 min, membranes were probed with primary antibodies against Gal-3 and HRP-conjugated secondary antibody. Immunoreactive proteins were detected using the enhanced chemiluminescence system. To control for protein loading, membranes were stripped and then incubated with β-actin monoclonal antibody.

### Statistical Analysis:

Statistical analyses were conducted with Student's t test. Values with P<0.05 were considered significant.

### Results

### Selection of DC-Binding Peptides.

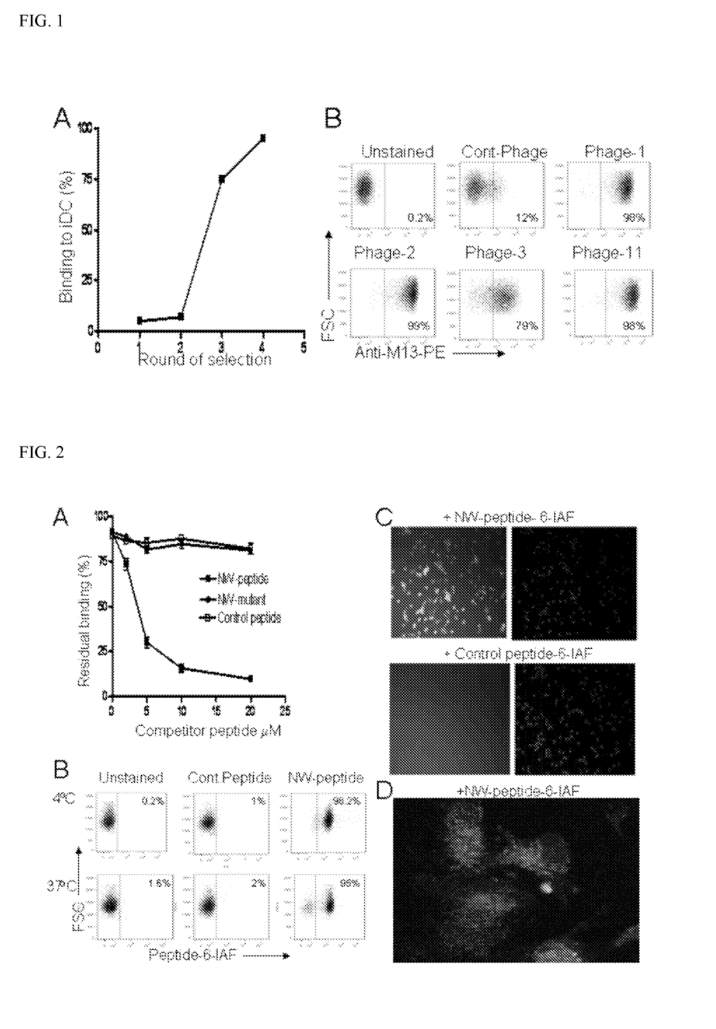

To identify novel DC-binding ligands, peptide phage libraries (Ph.D. 7-mer and 12-mer) were biopanned on iDCs. As shown in FIG. 1A, an exceptional enrichment of phage binders was obtained after three rounds of selection. The enrichment in early rounds demonstrates the selection of high affinity phages. Analysis of individual random phage clones from the fourth round of biopanning confirmed the strong binding of the selected phages to iDCs (FIG. 1B). Indeed, more than 90% of the amplified phage clones bound to iDCs. Positive phages consistently labeled most of the cells and the fluorescence intensities were always high.

In the next experiments, positive phage clones were amplified and analyzed by DNA sequencing to identify the peptide sequences (Table I). A single peptide NWYLPWLGTNDW (SEQ ID NO:17) (NW-peptide) dominated the sequences. The NW-peptide shares the motif NW-LPWL with peptide 2. Two clones displayed 7-mer short peptides. It should be noted that the binding intensity of phage-displaying the NW peptide (NW phage) was always high, suggesting the selection of a high affinity peptide.

### Specificity of the Phage-Displaying the NW-Peptide.

To characterize the binding specificity of the NW-phage, its binding potency to a panel of human cells was analyzed (Table II). The phage exhibited a strong binding to iDC, mDCs as well as to monocytes. Notably, the phage and the synthetic peptide bound to blood CD1c+ myeloid DCs and CD303+ plasmacytoid DCs. On the other hand, the phage did not bind to T cells, B cells, NK cells, human monocyte cell line THP-1, and all tested cancer cell lines. The phage binding to freshly isolated blood monocytes, but not to THP-1 cells underlies a significant difference between cancer cell lines and primary cells. Trypsine treatment of DCs eliminated phage binding, hence the receptor is a protein (data not shown).

To further investigate the importance of the NW peptide on phage binding, competition experiments were performed using synthetic peptides. The NW peptide, but not an irrelevant control peptide, effectively inhibited the phage binding to iDC in a concentration-dependent manner (FIG. 2A). The ability of the monovalent peptide to eliminate phage binding at low concentrations indicates the selection of high affinity peptide. The NW-peptide lacking the motif LPWL (NW-mutant) failed to reduce binding, thus this motif is important for the peptide interaction with its receptor.

In addition to competition experiments, the binding of 6-iodoacetamidofluorescein (6-IAF)-conjugated peptides to iDCs was assessed by flow cytometry. As shown in FIG. 2B, the NW peptide exhibited a strong binding both at 4° C. and 37° C., while no significant binding was obtained with control peptide. Most, if not all, cells bound the NW peptide. Peptide binding to DCs was also evaluated by epifluorescence microscopy (FIG. 2C). In contrast to control peptide, the NW peptide showed a strong cell staining Experiments using fluorescence confocal microscopy showed that the NW peptide was efficiently internalized by iDCs (FIG. 2D). Comparable data were obtained with mDCs. Therefore, Thus, the NW peptide can be used to target antigens to DCs.

### Peptide-Binding Did not Affect the Expression of Co-Stimulatory Molecules and DC Function

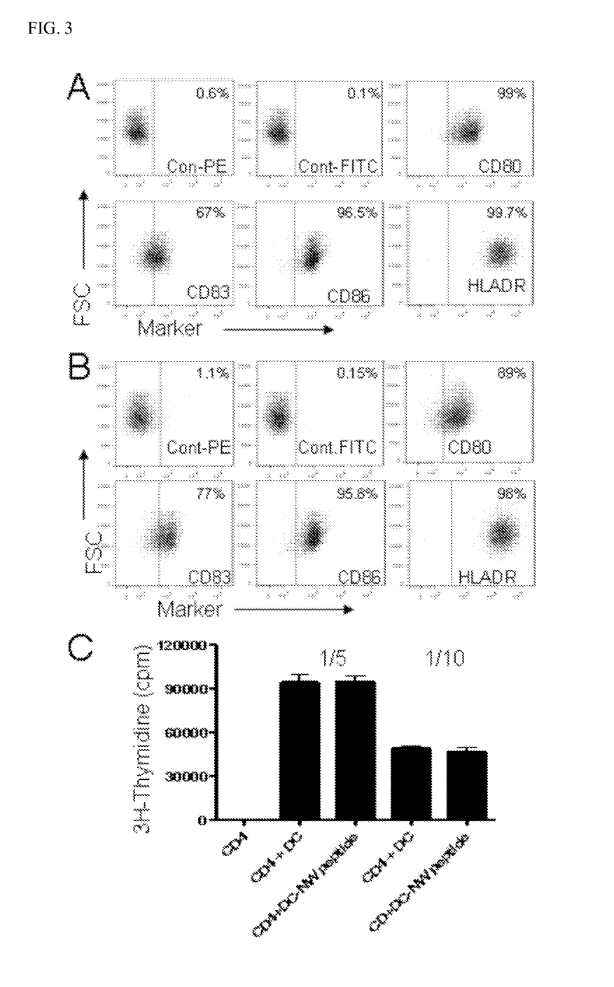

With respect to cancer immunotherapy and active immunization against infectious diseases, targeting moieties should not have a negative impact on DC immunogenic function. To investigate whether the NW peptide can modulate DC phenotype and function, mDC were incubated with the peptide for 48 h and subsequently the expression of CD80, CD83, CD86, and HLADR molecules were analyzed by flow cytometry. None of the analyzed markers were significantly affected by peptide binding (FIGS. 3A and B). The ability of mDCs to stimulate T-cell proliferation was assessed in a mixed leukocyte reaction (MLR), a hallmark of DC function. Untreated and peptide-treated mDCs induced comparable T-cell proliferation (FIG. 3C). The NW-peptide had no major negative effects on DC phenotype and function.

### NW Peptide can Mediate Protein Delivery to DCs.

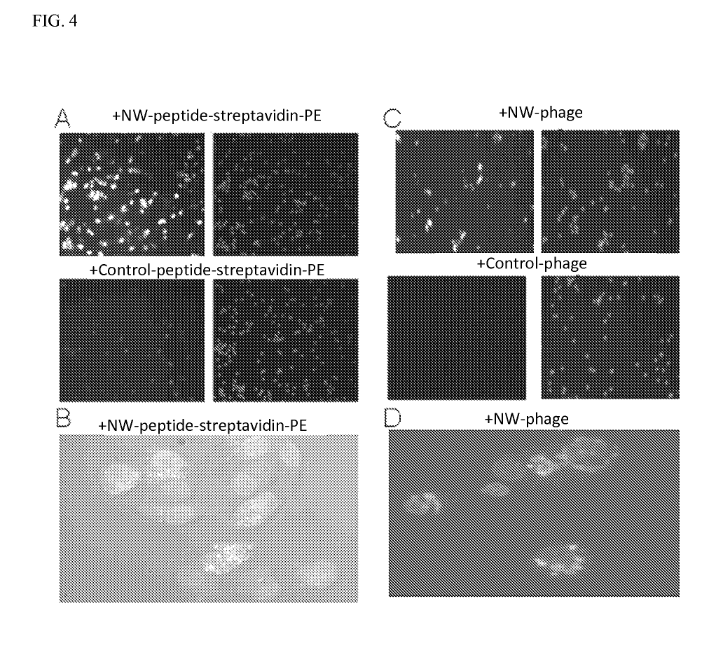

The use of the NW peptide to target antigens to DCs was assessed by examining its ability to promote the binding and uptake of streptavidin-PE complexes. For these experiments, streptavidin-PE was pre-incubated with either biotin conjugated NW peptide or biotin conjugated control peptide and then the mixtures were added to iDCs growing in Lab-Tek chamber slides, incubated at 4° C., washed and then transferred at 37° C. Since endocytotic processes are inhibited at 4° C., and streptavidin-PE does not bind to iDCs, the NW peptide-streptavidin-PE complexes can be internalized only after specific binding to iDCs. In contrast to control peptide, NW peptide mediated the binding of streptavidin-PE complexes to DCs (FIG. 4A). Confocal microscopy images showed a clear internalization and cellular localization of streptavidin-PE molecules (FIG. 4B). Similarly, the NW peptide was able to mediate the internalization of the phage particles into DCs (FIGS. 4C and D), supporting the delivery of large cargoes to DCs such as nanoparticles.

### NW-Peptide Promotes Binding of Pp65 Peptides to Dendritic Cells.

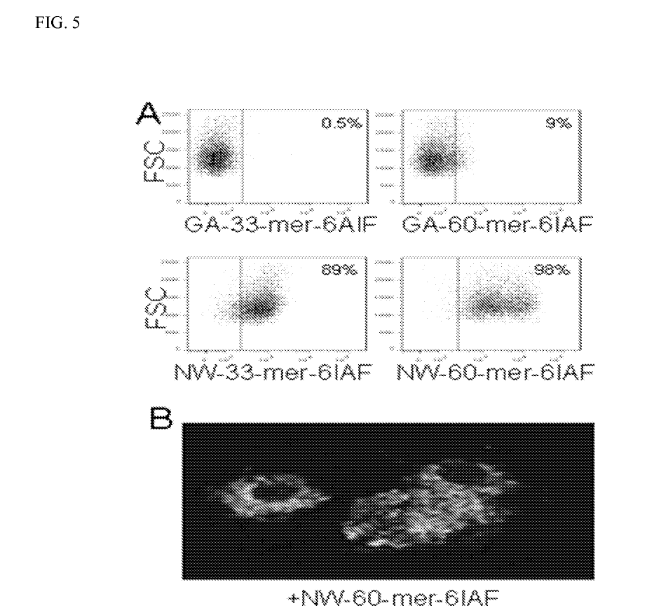

To assess whether the NW peptide could be used to direct foreign antigens to DCs, the CMV pp65 protein was used as a model antigen (Wills et al., (1996) J Virol. 70, 7569-7579). Long pp65 peptides were fused either to NW peptide (NW-33-mer, NW-60-mer) or to control peptide (GA-33-mer, GA-60-mer) were designed, conjugated to 6IAF and their binding to DCs was investigated by flow cytometry (FIG. 5A). Unlike the non-targeted pp65 peptides, The NW-pp65 fusion peptides bound to iDCs. Confocal microscopy analysis showed that cell-bound NW-pp65 peptide molecules were internalized (FIG. 5B). Similar results were obtained with the NW-33-mer peptide (data not shown).

### Targeting Pp65 Peptides to DCs Enhanced T Cell Proliferation.

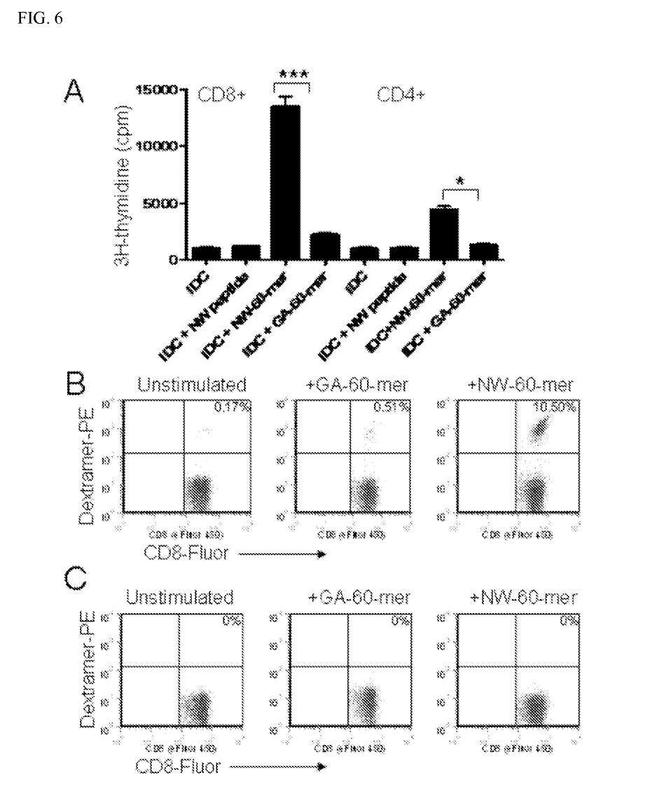

The capacity of the NW-pp65 fusion peptide (60-mer) to activate T cells from HLA-A2+/CMV positive donors was evaluated. It should be noted that the 48-mer pp65 peptide contains both MHC class I (e.g. NLVPMVATV) (SEQ ID NO:19) and class II (e.g. AGILARNLVPMVATV (SEQ ID NO:23), FFWDANDIYRI (SEQ ID NO:24)) epitopes, allowing the detection of CD4 and CD8 T-cell responses (24-26). For these experiments, semi mDCs were incubated with either targeted or untargeted pp65 peptide at 4° C., washed to remove unbound peptides, and then added to autologous purified CD4 or CD8 T cells. DCs were also incubated with the NW peptide only. All cultures were incubated at 37° C. for 5 days and T-cell activation was determined by measuring their proliferation potential (FIG. 6A). Targeting DCs with the NW peptide activated both CD8 and CD4 T cells, while no significant effect was obtained with untargeted pp65 peptide (GA-60-mer). Although the major route for presentation of exogenous antigens is via MHC class II molecules, the data indicate that antigen taken up by the NW peptide receptor also have access to the cytosol for MHC class I presentation to stimulate CD8 T cells. Dextramer staining of CD8 T cells specific for the NLVPMVATV (SEQ ID NO:19) epitope clearly showed the superior efficacy afforded by NW-targeted delivery to DCs (FIG. 6B). Indeed, cells incubated with the targeted pp65 peptide exhibited significantly higher activation potential than the corresponding untargeted peptide at equal molar concentrations (10.50% vs 0.51% P<0.001, n=3). Under the same conditions, no significant staining was obtained with HIV-dextramer (FIG. 6C).

Induction of CMV-Pp65-Specific T Cells from the Naïve Donor T-Cell Repertoire.

To evaluate whether the NW-pp65 fusion peptide, besides triggering memory responses, is able to activate naïve T cells, the fusion peptide was tested in an autologous in-vitro optimized culture conditions using PBMCs from HLA-A2+/CMV negative donors. With respect to naïve T-cell activation, some studies have demonstrated that the induction of primary responses is determined not only by the numbers antigen-specific precursors but also by the activation state of T regs. Indeed, depletion of CD45RO+ cells significantly enhanced the induction of primary virus-specific and anti-tumor T cell responses (Jedema et al., (2011). Haematologica. 96, 1204-12). CD45RO is expressed by memory T cells and T regs (FalciaBooth et al., (2010) J Immunol. 184, 4317-4326).

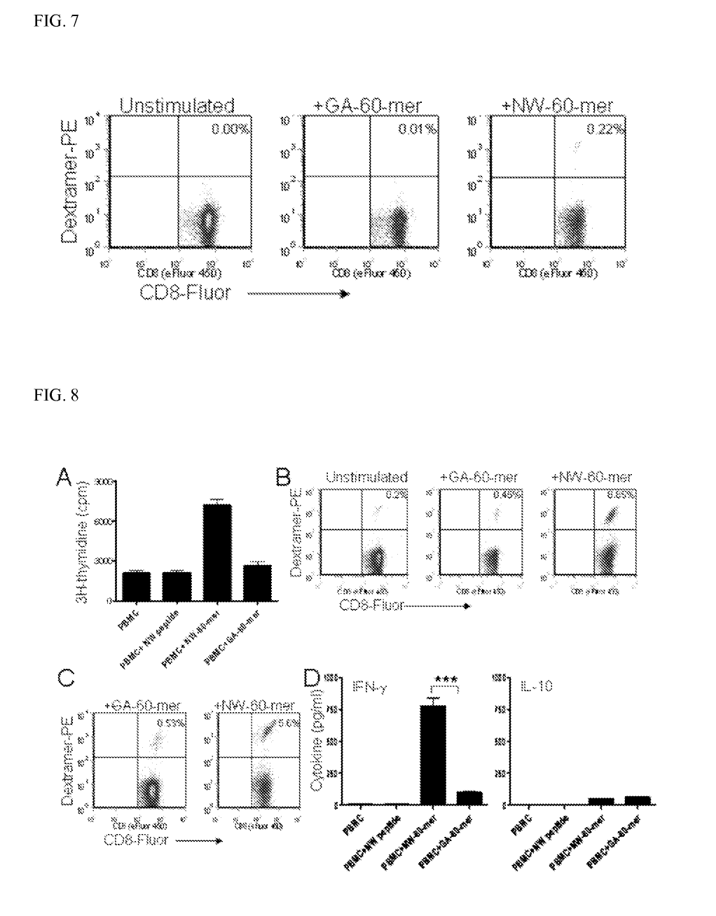

PBMCs from HLA-A2+/CMV negative donors were depleted of CD45RO+ cells and stimulated by repetitive co-culturing with NW-pp65 fusion peptide-targeted autologous DCs in the presence of human IL-7, which is important for in-vivo maintenance and expansion of the naïve T-cells (Fry, J Immunol. 174, 6571-6576). Autologous DCs were also incubated with untargated GA-pp65 peptide. As illustrated by a representative example in FIG. 7, the NW-pp65 fusion peptide activated pp65-specific naïve T cells when compared to the untargeted pp65 peptide (0.22% vs 0.01%). Thus, the NW peptide conjugated antigens can activate both memory and primary immune responses.

### Peptide Binding to Blood Monocytes Did not Hamper T-Cell Activation.

Monocytes have the capacity to differentiate into macrophages or inflammatory DCs in-vitro and in-vivo (Auffray et al., (2009) Annu Rev. Immunol. 27, 669-92). Given that the NW peptide bound to blood monocytes, it was investigated whether this binding affects T-cell activation. Therefore, proliferation assays were performed with whole PBMCs. The cells were incubated with the NW-pp65 or GA-pp65 fusion peptides at 4° C., washed to remove unbound peptides and then incubated at 37° C. for 5 days and cell proliferation was assayed by [³H]-thymidine incorporation (FIG. 8A). The targeted pp65 peptide stimulated T-cell proliferation, while untargeted peptide did not. To evaluate the extent to which CD8 lymphocyte proliferation was stimulated by the targeted peptide, dextramer staining was also performed (FIG. 8B). The CTL response was significantly enhanced in response to the targeted peptide as compared to non-targeted peptide (8.85% vs 0.45% P<0.001, n=3). When monocyte-depleted PBMCs were stimulated with the NW-pp65 fusion peptide, a strong CTL response relative to untargeted peptide was also obtained (FIG. 8C, 5.60% vs 0.53%), indicating that in the presence or absence of NW-peptide-binding monocytes, the NW-pp65 fusion peptide was able to activate T cells. In all experiments, no significant staining was obtained with HIV-dextramers.

Next, the immunostimulatory potential of the NW-pp65 fusion peptide was evaluated by assessing its ability to stimulate the overall IFN-γ and IL-10 production in PBMC cultures from CMV-positive donors (FIG. 8D). A significant increase in total amounts of secreted IFN-γ was obtained with the targeted peptide relative to untargeted peptide (P<0.001, n=4).

### DC-Targeting is Superior to Spontaneous Antigen Uptake.

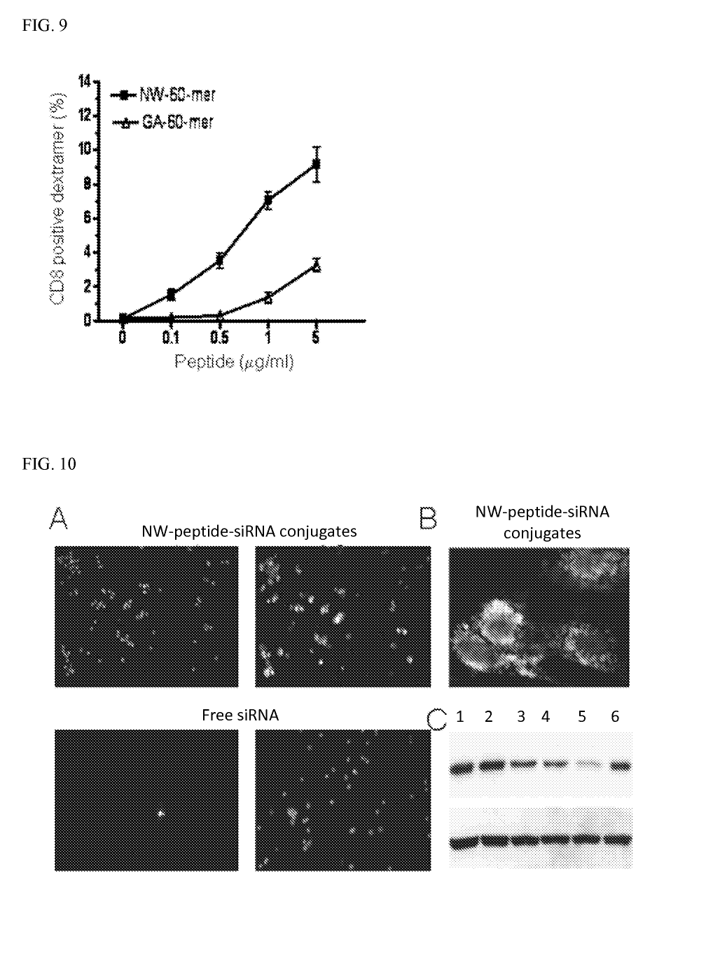

Since endocytosis was inhibited at 4° C., no significant effects of the untargeted pp65 peptides were anticipated because they do not bind specifically to DCs. Therefore, in the next experiments, the spontaneous uptake of untargeted and targeted pp65 peptides by DCs was compared. For these experiments, PBMCs were incubated at 37° C. with the peptides, washed to remove unbound peptides and further incubated at 37° C. for 12 days. Tetramer staining revealed a significant expansion of CD8 T cells by the NW-pp65 fusion peptide relative to untargeted peptide, particularly at lower peptide concentrations (FIG. 9). These data underlie the superiority of the NW peptide receptor to mediate antigen uptake over spontaneous uptake by blood APCs.

NW Peptide Facilitated siRNA Delivery to DCs.

One of the major challenges to the clinical development of gene silencing by small interfering RNA (siRNA) is its effective delivery to target cells (Whitehead et al., Nat Rev Drug Discov 8, 129-138). Given the effective internalization of NW peptide by DCs, its potential to direct siRNAs to DCs was evaluated. First, a fluorescence labeled siRNA targeting mouse IL-10 was covalently conjugated to the NW-peptide (NW peptide with GGGSC (SEQ ID NO:43) linker) through a thiol linkage and then the binding of the conjugates to iDCs was investigated by epifluorescence microcopy. In contrast to free siRNA molecules, the peptide siRNA conjugates bound to DCs (FIG. 10A). Furthermore, confocal microscopy analysis confirmed the intracellular delivery of the peptide-siRNA conjugates (FIG. 10B).

To demonstrate gene-silencing, the NW peptide was conjugated to 5′-thiol functionalized sense strand of a siRNA targeting human Gal-3, purified and then added to iDCs. A dose-dependent gene silencing response was evident after 48 h incubation time (FIG. 10C, lanes 3-5). By contrast, free siRNA or peptide molecules did not induce any detectable gene silencing at high concentrations (FIG. 10C, lane 2 and 6, respectively). These results indicate that biologically active siRNAs can be delivered by the NW peptide. Because siRNA molecules must be released into the cytoplasm in order to function (Elbashir et al., (2001) Nature 411, 494-498), the data further confirm the internalization of the peptide after binding to DCs.

### Deciphering the Structural Requirements for NW Peptide Binding to APCs.

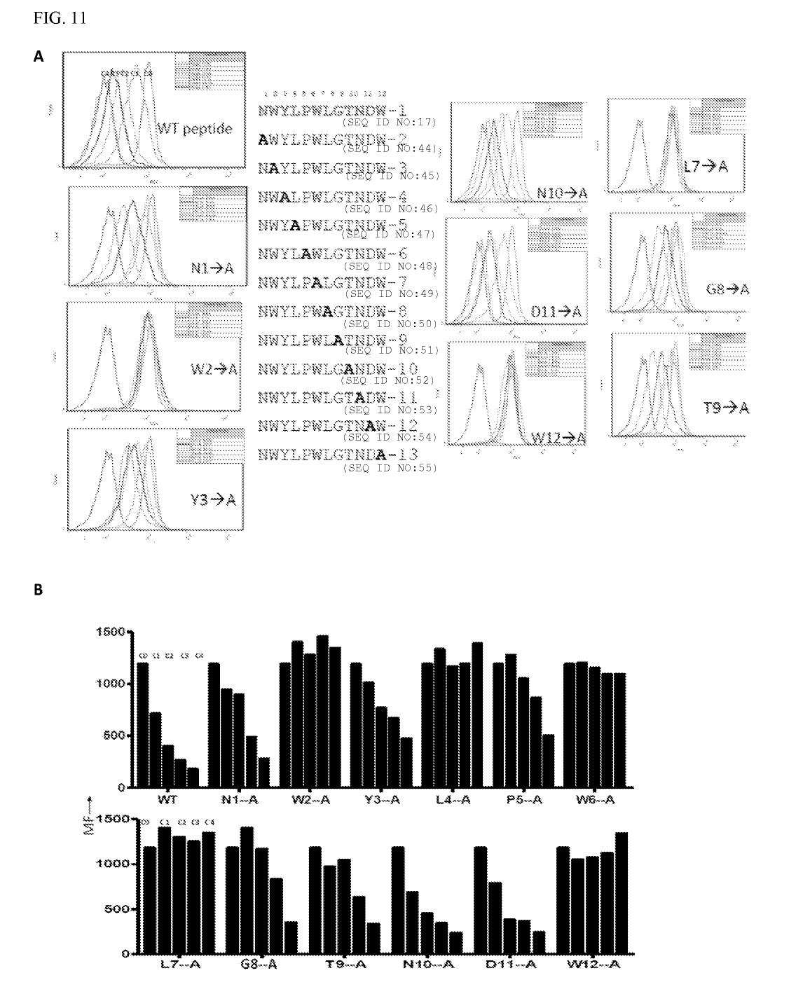

To uncover the contribution of individual side chains and identify which amino acids are responsible for the NW peptide binding to monocytes and dendritic cells, alanine scanning was performed. Derivatives of the NW peptide were synthesized with exchange of each amino acid by alanine (FIG. 11A). These peptide derivatives were used to compete with the NW phage binding to monocytes. For each peptide, four concentrations were tested. Bound phage particles were detected with the use of biotin conjugated anti-M13 antibody and PE-conjugated streptavidin. Representative examples of cytometric histograms are shown in FIG. 11A. The mean fluorescence intensities of PE positive cells are shown in FIG. 11B. The NW peptide (WT) competed effectively with the phage binding to monocytes with an IC₅₀ of approximately 0.5 uM. The replacement of tryptophan (W) at position 2, 6, or 12 by a single alanine abolished the binding. Indeed, no significant competition with the phage binding was seen even at high peptide concentrations (C4=20 uM). Similarly, replacement of leucine (L) at position 4 or 7 by alanine inhibited the peptide binding (FIG. 11B). These results indicate that both W and L are important for peptide binding to its receptor expressed by antigen presenting cells such as monocytes and DCs.

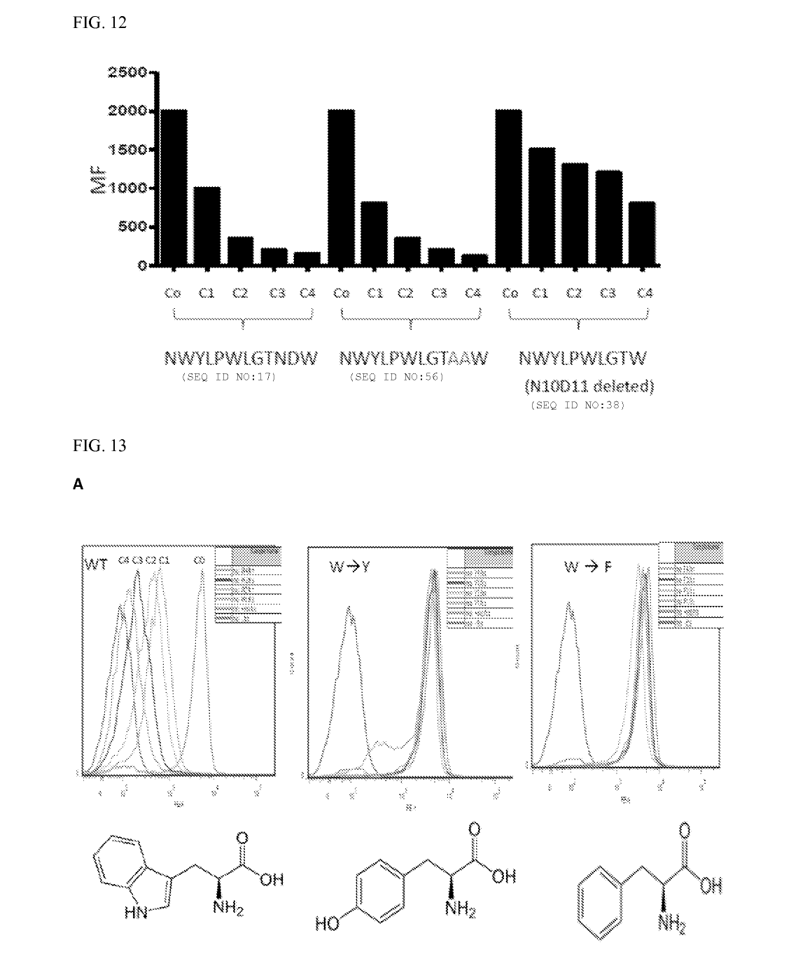

In contrast, replacement of asparagines (N) at position 10 and aspartic acid (D) at position 11 with a single alanine did not affect the binding of the mutant peptides. Indeed, these mutant peptides effectively competed with the NW phage binding to monocytes with IC50 comparable to that of the wild type peptide (FIG. 11B). As shown in FIG. 11B, position-specific replacement of the other amino acids by alanine reduced but did not abolish peptide binding. Like the single alanine mutation alone, N10D11 double mutant also effectively competed with the binding of the NW phage to monocytes (FIG. 12). The finding that the peptides with mutations at N10 and/N11 exhibits wildtype activity demonstrates that the amino acids N10 and D11 are not required for the NW peptide binding. Notably, the mean fluorescence of the double mutant at C3 concentration is compared to that of unstained cells (250). Deletion of N10D11 reduced the binding, as seen by the approximately 20-fold reduction in IC₅₀. Thus, the distance between residues threonine (T9) and W12 participating in peptide binding is important for the formation of the active peptide structure.

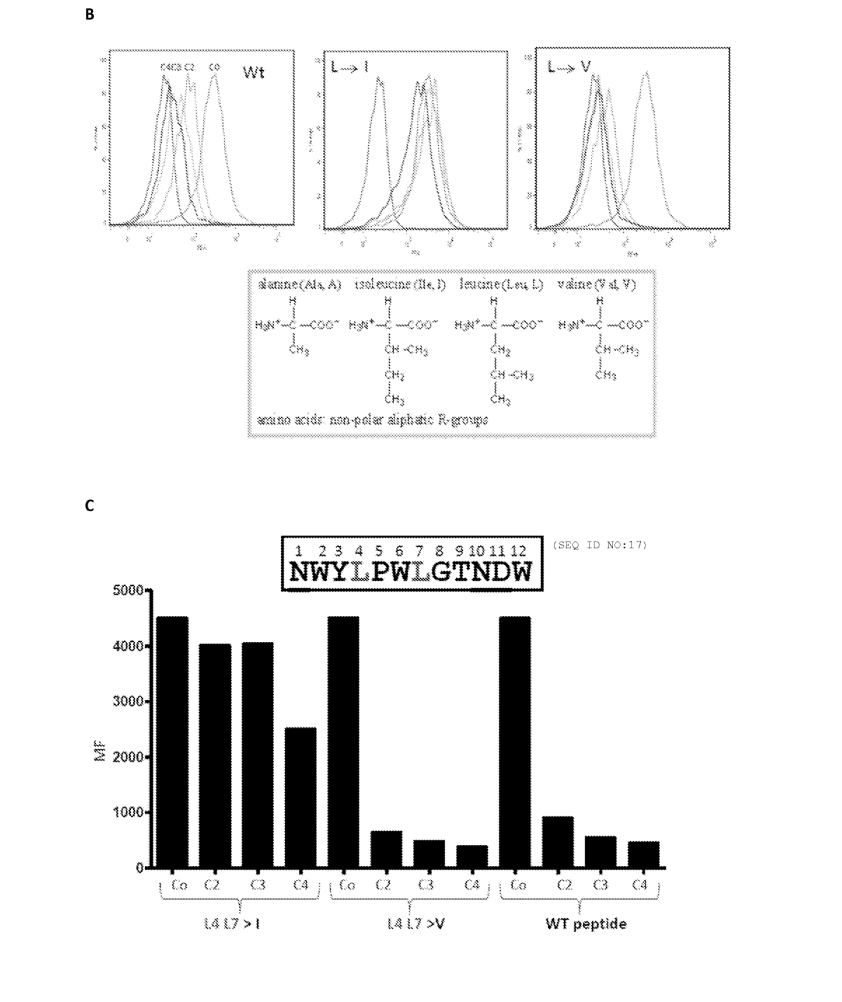

The effects of more conservative replacement of W and L residues found to be sensitive to replacement by alanine was investigated. Replacement of tryptophan by phenylalanine or tyrosine resulted in complete inhibition of peptide binding even at high concentrations (FIG. 13A). Therefore, the side chain of tryptophan (indole ring) is important for peptide binding. On the other hand, replacement of leucines L4 and L7 with valine (V) did not affect peptide binding, while replacement with isoleucine (I) only partially inhibited peptide binding (FIG. 13B, 13C). Thus, it appears that while W2, W6 and W12 are essential, the L4 and L7 tolerate certain conservative modifications, particularly from L to V.

### Targeting Mart-1 Antigen to Blood APCs.

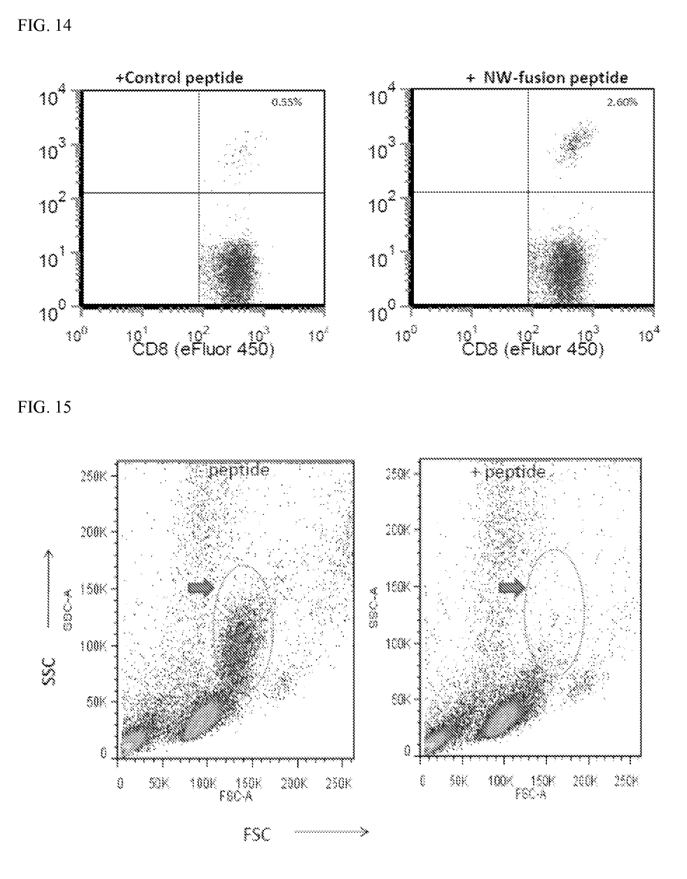

With respect to antigen targeting, short peptides present an attractive alternative to antibodies. Due to their small size peptides have improved pharmacokinetic properties, characterized by higher effectiveness of tumor penetration. Furthermore, peptides do not possess the immunogenic potential of antibodies, while they are easier and cheaper to synthesize and conjugate to desired molecules. To further evaluate the potential of the NW peptide to target antigens to blood APCs, the NW was fused to a long Mart-1 peptide, which contains the HLA0201-restricted CTL epitope (EAAGIGILTV). Peripheral blood mononuclear cells from a metastatic melanoma patient were incubated with the NW-fusion peptide or control peptides at 4 C for 1 h, the cells were washed to removed unbound peptides and then incubated at 37° C. for 7 days. Subsequently, T-cell proliferation was evaluated by MART-1 tetramer staining of CD8+ T-cell population (FIG. 14). In contrast to control peptide, the NW peptide enhanced T-cell proliferation (0.55% vs 2.6%).

Depletion of Monocytes from PBMC Via the NW Peptide.

Given the binding of the NW peptide to monocytes, its use in magnetic cell separation techniques was investigated. In general, these methods are based on the attachment of small magnetic particles to cells via antibodies. When the mixed population of cells is placed in a magnetic field, those cells that have beads attached will be attracted to the magnet and may thus be separated from the unlabeled cells. In these experiments, peripheral blood mononuclear cells were isolated from buffy coats of healthy donors by density gradient centrifugation. Around 10⁶ cells were incubated with biotin labeled NW peptide (10 μg) for 30 min at 4° C. with rocking in PBS buffer supplemented with 1% FCS (binding buffer). Subsequently, the cells were washed with the binding buffer to remove unbound peptide and then they were suspended in 100 μl binding buffer. Subsequently, pre-washed Dynabeads M-280 streptavidin (20 μl) were added and the mixtures were incubated for 15 min at room temperature with agitation. After, the mixtures were placed in the separation magnet, during which time the beads and any attached cells are drawn to one side of the tube. Non-attached cells were carefully aspirated off and analyzed by flow cytometry to check the removal of the monocyte fraction (FIG. 15). The data show that most, if not all monocyte population were removed, supporting the use of the NW peptide and derivatives in magnetic separation techniques.

In summary, the current study shows that the NW peptide can be used to efficiently deliver various molecules to DCs. Targeting of CMV pp65 peptides to DCs resulted in strong T-cell responses relative to untargeted peptides. Moreover, the NW peptide was able to deliver siRNAs to DCs and gene silencing was achieved.

TABLE I
Binding and amino
acid sequences of the selected phages

| Phage | Peptide sequence | Frequency | Binding | |
|---|---|---|---|---|
| DC2 | NWYLPWLGTNDW | 24/30 | ++++ | (SEQ ID |
| | | | | NO: 17) |
| DC6 | QWELPWLMQPPL | 2/30 | ++++ | (SEQ ID |
| | | | | NO: 25) |
| DC13 | SPGLSLVSHMQT | 2/30 | +++ | (SEQ ID |
| | | | | NO: 26) |
| DC10 | QLPRTAL | 1/30 | +++ | (SEQ ID |
| | | | | NO: 27) |
| DC19 | GETRAPL | 1/30 | +++ | (SEQ ID |
| | | | | NO: 28) |

Binding of the phage clones to iDC was analyzed by flow cytometry.
Binding >80-100%: ++++, binding <80%: +++.

TABLE II
Analysis of the phage and peptide binding to human cells.

| | Phage | Peptide |
|---|---|---|
| Cell type | Binding | Binding |
| iDCs | ++++ | ++++ |
| mDCs | ++++ | ++++ |
| Blood myeloid DCs | ++++ | ++++ |
| Blood pDCs | ++++ | ++++ |
| Monocytes | ++++ | ++++ |
| T cells | − | − |
| B cells | − | − |
| NK cells | − | − |
| THP-1 | − | − |
| Cancer cell lines* | − | − |
| Normal HMEC | − | − |

pDC, plasmacytoid., THP-1, human monocytic leukemia cell line., HMEC, human mammary epithelial cells.
*The following cancer cell lines were tested: Human breast cancer cell lines (MCF-7, MDA-MB 231), colon cancer cell line SW480 and acute lymphoblastic leukemia cell line REH.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

1. A polypeptide comprising: an antigen presenting cell targeting peptide having the amino acid sequence X₍ₙ₎LPWLX(ₘ₎ (SEQ ID NO:13), wherein X is any amino acid, and m and n are integers.

2. The polypeptide of claim 1, wherein said antigen presenting cell targeting peptide has the amino acid sequence XWYLPWLG (SEQ ID NO:14) or XWYLPWLGTNDW (SEQ ID NO:15), wherein X is any amino acid.

3. The polypeptide of claim 1, wherein said antigen presenting cell targeting peptide has the amino acid sequence NWYLPWLG (SEQ ID NO:16) or NWYLPWLGTNDW (SEQ ID NO:17).

4. The polypeptide of claim 3, wherein said antigen presenting cell targeting peptide has the amino acid sequence NWYLPWLGTNDW (SEQ ID NO:17).

5. The polypeptide of claim 1, wherein said antigen presenting cell targeting peptide has the amino acid sequence selected from the group consisting of XWYLPWLGTNDW (SEQ ID NO:15), NWXLPWLGTNDW (SEQ ID NO:30), NWYLXWLGTNDW (SEQ ID NO:31), NWYLPWLXTNDW (SEQ ID NO:32), NWYLPWLGXNDW (SEQ ID NO:33), NWYLPWLGTXDW (SEQ ID NO:34), NWYLPWLGTNXW (SEQ ID NO:35), NWYLPWLGTNW (SEQ ID NO:36), NWYLPWLGTDW (SEQ ID NO:37), and NWYLPWLGTW (SEQ ID NO:38), wherein X denotes any amino acid.

6. The polypeptide of claim 1, wherein said antigen presenting cell targeting peptide has the amino acid sequence selected from the group consisting of NWY_{z}PWLGTNDW (SEQ ID NO:39), NWYLPW_{z}GTNDW (SEQ ID NO:40) and NWY_{z}PW_{z}GTNDW (SEQ ID NO:31), wherein z is an amino acid with a hydrophobic branched aliphatic side chain.

7. The polypeptide of claim 1, wherein said antigen presenting cell targeting peptide has the amino acid sequence XWYLPWLG (SEQ ID NO:14) or XWYLPWLGTNDW (SEQ ID NO:15), wherein X is any amino acid.

8. The polypeptide of claim 1, wherein said peptide is linked to a molecule of interest.

9. The polypeptide of claim 8, wherein said molecule of interest is selected from the group consisting of an antigen, an antisense compound, an aptamer, and an siRNA.

10. The polypeptide of claim 9, wherein said antigen is a cancer antigen or a foreign antigen.

11. The polypeptide of claim 1, wherein said peptide further comprises sequence selected from the group consisting of LTVSPWY (SEQ ID NO:18) and a cationic peptide.

12. The polypeptide of claim 11, wherein said cationic polypeptide is RRRRRRRRR (SEQ ID NO:42).

13. The polypeptide of claim 8, wherein said antigen presenting cell targeting peptide and said molecule of interest are in a fusion polypeptide.

14. The polypeptide of claim 8, wherein said antigen presenting cell targeting peptide and said molecule of interest are complexed or non-covalently linked.

15-21. (canceled)

22. A composition comprising the polypeptide of claim 1, and a second component selected from the group consisting of an adjuvant and a pharmaceutically acceptable carrier.

23-25. (canceled)

26. A method of inducing an immune response, comprising:
administering the composition of claim 22 to a subject, wherein said administering induces an immune response against said molecule of interest.

27. The method of claim 26, wherein said immune response is a T-cell mediated immune response.

28. The method of claim 27, wherein said immune response is against a cancer cell.

29. The method of claim 27, wherein said immune response is against a foreign antigen.

30. A method of gene silencing in antigen presenting cell, comprising:
administering the composition of claim 22 to a subject, wherein said administering results in gene silencing in said antigen presenting cell.

31. (canceled)

32. A method of identifying a cell that binds to the peptides of claim 1, comprising:
a) contacting a solid support comprising a polypeptide affixed to a solid support with a sample; and
b) identifying or purifying cells that bind to said polypeptide.

33. (canceled)
